(19) United States
(12) Patent Application Publication
(10) Pub. No.: US 2019/0167576 A1
(43) Pub. Date: Jun. 6, 2019

(54) USE OF HIGH AFFINITY MONOCLONAL ANTIBODY PRODUCT BINDERS TO INCREASE THE DURATION OF ACTION OF THERAPEUTIC MONOCLONAL ANTIBODY PRODUCTS IN A BIOLOGIC TISSUE

(71) Applicant: The Board of Trustees of the Leland Stanford Junior University, Stanford, CA (US)

(72) Inventor: Ira Hyman Schachar, Palo Alto, CA (US)

(21) Appl. No.: 16/325,279

(22) PCT Filed: Sep. 6, 2017
(86) PCT No.: PCT/US2017/050330

§ 371 Date: Feb. 13, 2019

Related U.S. Application Data
(60) Provisional application No. 62/506,485, filed on May 15, 2017.
(60) Provisional application No. 62/441,709, filed on Jan. 3, 2017.
(60) Provisional application No. 62/384,720, filed on Sep. 8, 2016.

(51) Int. Cl. A61K 9/00; A61P 27/02; C07K 14/31; C07K 14/315; C07K 14/705; C07K 16/22; A61K 47/68; A61K 31/155; A61K 47/66

(52) U.S. Cl. CPC A61K 9/0048; A61P 27/02; C07K 14/31; C07K 14/315; A61K 47/66; C07K 16/22; A61K 47/68; A61K 31/155; C07K 14/705

(57) ABSTRACT

An anchor molecule is provided that (1) binds monoclonal antibody products with high affinity and (2) is anchored in a biologic tissue of interest. Therapeutic antibodies can either be delivered to the biologic tissue before, during, or after the delivery of the anchor molecule. Because the present invention binds monoclonal antibody products and is anchored in a biologic tissue, the result is that monoclonal antibody products are also anchored in a biologic tissue.

### CROSS REFERENCE

This application is a 371 application and claims the benefit of PCT Application No. PCT/US2017/050330, filed Sep. 6, 2017, which claims benefit of U.S. Provisional Patent Application Nos. 62/506,485, filed May 15, 2017, 62/441,709, filed Jan. 3, 2017 and 62/384,720, filed Sep. 8, 2016, which applications are incorporated herein by reference in their entirety.

### BACKGROUND

Since the original FDA approval of a therapeutic monoclonal antibody in 1986, there has been a steady increase in the number of therapeutic monoclonal antibodies, Fc-fusion proteins, antibody fragments, and antibody-drug conjugates (collectively known as monoclonal antibody products). Due to their specificity, these monoclonal antibody products have rapidly become a leading product of the pharmaceutical industry, treating a wide variety of disease from cancer to inflammation, intraocular neovascularization to rare orphan conditions. Currently there are over 50 monoclonal antibody products that have been FDA approved and at the current rate of development this is expected to grow by 4 to 5 per year, for the foreseeable future. Despite their incredible efficacy and specificity, monoclonal antibody products have significant drawbacks. Chief among these is their relatively high production costs combined with relatively short half-life in biologic tissue. The net result is expensive therapeutics that need to be frequently reinjected, rapidly driving up costs for patients and health systems worldwide.

These challenges are particularly problematic for ocular diseases. Macular degeneration (AMD), diabetic retinopathy, and retinal vein occlusions are the leading causes of blindness worldwide. The prevalence of AMD is expected to surpass 200 million worldwide in the next 5 years, diabetic retinopathy already affects over 100 million people and may triple over the coming twenty years, and retinal vein occlusions affect over 15 million people and is expected to increase as the prevalence of hypertension increases. Upregulation of vascular endothelial growth factor (VEGF) is a key component in the disease pathogenesis in AMD, diabetic retinopathy, and retinal vein occlusion. Monoclonal antibody products, which target and neutralize VEGF have been remarkably successful at slowing disease progression. Further research have identified other potential factors causing disease pathogenesis such as platelet derived growth factor (PDGF), complement factor H (CFH), alternative isoforms of VEGF, and human factor VII. Monoclonal antibody products directed against these new targets are actively in clinical trials and will likely only expand the number of patients receiving intravitreal therapeutic monoclonal antibody products.

Unfortunately, therapies require injection of the antibody into the vitreous cavity of the eye as these molecules cannot reach the eye at sufficient concentrations when delivered systemically. More troublesome, these antibodies are rapidly cleared from the vitreous cavity, resulting in half-lives of days to at most weeks. Such short half-lives result in the need for repeated intraocular injections occurring every 4-8 weeks, sometimes for years. Frequent injections not only carry increased risk of devastating eye infection, but also put an enormous burden on patients and on the health care industry. In fact, the use of anti-VEGF agents for ophthalmology are currently the single biggest Medicare drug expenditure. A method of increasing resident time of not only anti-VEGF agents, but also any therapeutic antibody product injected into the eye is needed.

Attempts at reducing this burden have focused on modifying monoclonal antibody products, such as adding polyethylene glycol (PEGylation), or embedding the monoclonal antibody product in a hydrogel to decrease the rate of clearance from biologic tissue. Unfortunately, both of these attempts have met with limited success. The present invention provides an alternative solution.

### SUMMARY

Compositions and methods are provided for increasing the duration of action of therapeutic antibodies, e.g. monoclonal antibodies, and derivatives thereof when administered to a biologic tissue. A modular approach is provided, in which a biologic “anchor” (also referred to herein as a anchored high affinity antibody product binder) molecule is delivered to the biologic tissue, which anchor acts to retain a monoclonal antibody in the tissue, thereby increasing the duration of action. The duration of action of the therapeutic antibody is increased relative to the duration of action in the absence of the anchor molecule. Anchoring as used herein refers to a method of reducing the rate of clearance of the therapeutic antibody from the biologic tissue of interest.

A benefit of the present invention is the ability to utilize one product for the retention of any therapeutic antibody. In some embodiments the therapeutic antibody comprises an IgG Fc region. In some embodiments, the Fc region is a human IgG, including without limitation one or more of human IgG1, IgG2a, IgG2b, IgG3, IgG4, etc. In some embodiments the biologic tissue is the vitreous cavity of the eye. In some embodiments, the anchor is not fused, covalently linked or conjugated to a therapeutic monoclonal antibody.

An anchor molecule comprises two functionalities: (1) binding monoclonal antibody products with a dissociation constant (K_{D}) less than 10⁻⁶ M (K_{D}<10⁻⁶ M), and (2) anchoring functionality for the biologic tissue of interest. In some embodiments the monoclonal antibody binding function comprises a region or moiety that provides for high affinity binding to an antibody, including without limitation an antibody Fc region, e.g. an antibody IgG Fc region. The affinity of the anchor for an Fc region may have a dissociation constant (K_{D}) less than about 10⁻⁶ M, less than about 10⁻⁷ M, less than about 10⁻⁶ M, less than about 10⁻⁹ M, or less. In some embodiments the dissociation constant (K_{D}) is less than about 10⁻⁷ M. In some embodiments the anchor is a polypeptide, including without limitation protein A or protein G and derivatives and fragments thereof. In some such embodiments a protein A derivative is a minimized Z domain polypeptide. In other embodiments the anchor is a non-peptide molecule, including without limitation triazine-based molecules, as described herein.

The anchoring functionality can be provided in various different configurations. In some embodiments anchoring is achieved by a region or moiety that binds to a molecule present in the biologic tissue, where the region or moiety may be a peptide, or a non-peptide. In some such embodiments, the molecule is hyaluronic acid, for example where a polypeptide anchor comprises a moiety that binds to hyaluronic acid at a high affinity, which moiety may comprise or consist essentially of, for example, a poly-arginine sequence, a poly-lysine sequence, a guanidinium group, a LINK domain, an HA binding peptide, etc. In other embodiments the anchor comprises an anchoring moiety or group that retains the anchor in the biological tissue of interest, e.g. where the anchor comprises a polymer that increases tissue retention. Polymers may include, for example, chitosan, polymethacrylate microbeads, an aldehyde modified hyaluronic acid, maleimide-modified hyaluronic acid, etc.

In some embodiments, methods are provided for treating an individual with a therapeutic antibody, the method comprising administering a combination of an effective dose of an anchor, as described herein, to the targeted biologic tissue, with a therapeutic monoclonal antibody, where the therapeutic antibody can be delivered to the biologic tissue before, during, or after the delivery of the anchor. Generally the anchor is delivered directly to the targeted tissue. In some embodiments, the targeted tissue is the vitreous cavity of the eye. In some embodiments, the therapeutic antibody is also delivered directly to the targeted tissue, e.g. the vitreous cavity of the eye. In some embodiments the methods provided herein allow for retaining biological activity of the monoclonal antibody while reducing the frequency of administration. For example the period of time between administrations, while allowing for an effective dose of a therapeutic antibody, may be reduced by about 25%, by about 50%, by about 75%, by about 100%, by 2-fold, 3-fold or more. For treatment of chronic or long term conditions, the anchor may be administered multiple times as needed.

In another embodiment of the invention, an article of manufacture containing materials useful for the treatment of the conditions described above is provided. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). The active agent in the composition can be an anchor molecules as described herein. The label on, or associated with, the container indicates that the composition is used for treating the condition of choice. Further container(s) may be provided with the article of manufacture which may hold, for example, a therapeutic antibody. The article of manufacture may further include other materials desirable from a commercial and user standpoint, including a pharmaceutically-acceptable buffer, such as phosphate-buffered saline, Ringer's solution or dextrose solution, other buffers, diluents, filters, needles, syringes, and package inserts with instructions for use.

Because the present invention binds monoclonal antibody products and the present invention is anchored in a biologic tissue, the result is that monoclonal antibody products which bind to the present invention are also anchored in the biologic tissue. The net result is that through this system of binding monoclonal antibody products and anchoring to the biologic tissue, monoclonal antibody products are retained in the biologic tissue thereby increasing their duration of action.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 provides an overview. A high affinity monoclonal antibody product binder (anchor) with a dissociation constant (K_{D}) less than 10₋₆ M (K_{D}<10₋₆ M) is anchored in a biologic tissue. Because the high affinity monoclonal antibody product binder is anchored in the biologic tissue, it does not readily diffuse from the tissue and through its binding to a monoclonal antibody product, the monoclonal antibody product is also retained in the biologic tissue.

FIG. 2: A schematic of naturally occurring Protein A, which is one example of a naturally occurring monoclonal antibody product binder. Protein A consists of six domains. Domain X is located in the bacterial cell wall. There are five extracellular domains, A through E. Each domain is capable of binding a monoclonal antibody product through either an Fc binding domain or a F(ab) binding domain.

FIG. 3: Soluble native Protein A is one example of a naturally occurring monoclonal antibody product binder. Soluble native Protein A is devoid of the X domain. Soluble native Protein A contains both Fc and FAB binding domains. FAB region of a monoclonal antibody product binds to the FAB binding domain of soluble native Protein A (sometimes referred to as alternative binding), while Fc region of a monoclonal antibody product binds to the Fc binding domain. Monoclonal antibody products which bind to soluble Protein A through the FAB binding region may elicit immune cell activation.

FIG. 4: Any protein that is based at least in part on the primary, secondary, tertiary, or quaternary structure of native monoclonal antibody product binder is considered a recombinant form of the native monoclonal antibody product binder and is included in the present disclosure. Of particular interest are fragments and variants of Protein A. One example of a recombinant form of a naturally occurring monoclonal antibody product binder is illustrated. Soluble native Protein A has 5 domains each containing both Fc binding regions and FAB binding regions. Here, a recombinant form of Protein A is illustrated that is devoid of the FAB binding domains.

FIG. 5: Recombinant Protein A can be a fragment or variant of Protein A, for example as set forth in Table 1. Recombinant Protein A can have multiple monoclonal antibody product binding domains or a single monoclonal antibody product binding domain. Recombinant Protein A can have an expanded sequence, a modified sequence, or a minimized sequence in order to change certain key characteristics for the purpose of optimizing the effectiveness of the present invention.

FIG. 6: A form of Recombinant Protein A comprises or consists essentially of the Z-domain, which is a synthetic domain based upon the sequences of the five domains (A-E) of native Protein A. The Z-domain (as well as native binding domains of Protein A), contains key alpha helices responsible for monoclonal antibody binding. An exemplary sequence is set forth in SEQ ID NO:3, VDNKFNKEQQNAFYEILHLPNLNEEQRNAFIQSLKDDPSQSANLLAE AKKLNDAQAPK.

FIG. 7: A form of recombinant Protein A that has undergone a combination of structure-based design and affinity maturation methods to iteratively improve the stability and binding affinity of the two alpha-helices, while also decreasing the length of the amino acid sequence is provided in SEQ ID NO:1 FNMQQQRRFYEALHDPNLNEEQRNAKIKSIRDD. Variants of this sequence are provided in the compositions.

FIG. 8 depicts an immunoglobulin that binds monoclonal antibody products. This is an example of a high affinity monoclonal antibody product binder that is based on an immunoglobulin. Both immunoglobulins and fragments thereof can provide high affinity monoclonal antibody product binders.

FIG. 9 illustrates generalized structures of high affinity non-immunoglobulin affinity scaffolds. For each of these scaffolds, a scaffold library consisting of many sequence variants is constructed. The library undergoes biopanning against one or more monoclonal antibody products. The subsequent sequences can then undergo one or more rounds of structure based design or affinity maturation to further enhance binding to monoclonal antibody products. The present disclosure includes any high affinity binder that is based on any of these generalized structures to generate a high affinity monoclonal antibody binder.

FIG. 10: An example of a stepwise approach of creating a high affinity monoclonal antibody binder from a non-immunoglobulin affinity scaffold. A non-immunoglobulin affinity scaffold of many sequence variants is created. Biopanning is then used to identify the subset of sequences that bind monoclonal antibody products. Serial rounds of structure based design and affinity maturation are undertaken to further enhance binding affinity to generate a high affinity monoclonal antibody binder.

FIG. 11: Synthetic non-peptide based compounds can provide a high affinity monoclonal antibody product binder. One example of such synthetic non-peptide based high affinity monoclonal antibody product binders are triazine-based compounds.

FIG. 12. Triazine based high affinity monoclonal antibody binding agents.

FIG. 13. Triazine based high affinity monoclonal antibody binding agents.

FIG. 14. Triazine based high affinity monoclonal antibody binding agents.

FIG. 15A-15B: Examples of synthetic non-peptide based compounds that can provide high affinity monoclonal antibody product binders.

FIG. 16: High affinity monoclonal antibody product binders can comprise one or more binding domains, e.g. 2, 3, 4 or more binding domains. Such fusion compounds can be composed of similar or dissimilar high affinity monoclonal antibody products binders.

FIG. 17: An anchoring substance is a region or moiety that acts to retain the anchor in a tissue of interest by providing resistance to one or both of diffusion and degradation from a biologic tissue. When the anchoring substance is inserted into a biologic tissue it remains in the biologic tissue for an extended period of time due to either slow degradation, slow diffusion, or both slow degradation and slow diffusion rates out of the tissue.

FIG. 18: A high affinity monoclonal antibody product binder is linked to an anchoring substance that resists degradation, restricts diffusion, or does both. When the high affinity monoclonal antibody product binder linked to the anchoring substance is inserted into a biologic tissue, the anchoring substance is retained in the biologic tissue along with the high affinity monoclonal antibody product binder.

FIG. 19: An aldehyde-modified hyaluronic acid is an anchoring substance. Aldehyde-modified hyaluronic acid is linked via an amide linkage to the antibody binding domain, exemplified here as a minimized version of the Z-domain of Protein A.

FIG. 20: A maleimide-modified hyaluronic acid is an anchoring substance. Maleimide-modified hyaluronic acid is linked via a thioether linkage to the antibody binding domain, exemplified here as a minimized version of the Z-domain of Protein A.

FIG. 21. Multiple domains of similar or dissimilar high affinity monoclonal antibody product binders are linked together such that the resultant fusion protein has sufficient size, charge, polarity or hydrophobicity to resist degradation or restrict diffusion from a biologic tissue. The resultant high affinity monoclonal antibody binder thus remains anchored in a biologic tissue and is able to bind and retain therapeutic monoclonal antibody products.

FIG. 22. A high affinity monoclonal antibody product binder is linked to an active-crosslinker. When the high affinity binder/active cross-linker is inserted into a biologic tissue, the active cross-linker forms a covalent attachment to the biologic tissue. The result is a high affinity monoclonal antibody product binder that is covalently attached to the biologic tissue and is able to bind and retain monoclonal antibody products.

FIG. 23: A high affinity monoclonal antibody product binder is linked to a substance that non-covalently binds to biologic tissue. When the high affinity binder/noncovalent binding substance is inserted into a biologic tissue, the high affinity monoclonal antibody binder is anchored in the biologic tissue through the non-covalent binding of the noncovalent binding substance and is able to bind and retain monoclonal antibody products.

FIG. 24: An affibody that binds to monoclonal antibody products with a dissociation constant (K_{D}) less than 10⁻⁶ M (K_{D}<10⁻⁶ M) is linked with a peptide linker to an affibody that binds non-covalently to biologic tissue.

FIG. 25: A high affinity monoclonal antibody product binder with a method of anchoring is inserted into the vitreous cavity of the eye. Monoclonal antibody products that are subsequently inserted into the vitreous cavity are retained in the vitreous cavity through binding to the high affinity monoclonal antibody product binder that is anchored in the vitreous thereby increasing the resident time and duration of action of monoclonal antibody products in the eye.

FIG. 26: A high affinity monoclonal antibody product binder is linked to an anchoring substance is inserted into the vitreous cavity of the eye. Monoclonal antibody products that are subsequently inserted into the vitreous cavity are retained in the vitreous cavity through binding to the high affinity monoclonal antibody product binder that is anchored via the anchoring substance in the vitreous thereby increasing the resident time and duration of action of monoclonal antibody products in the eye.

FIG. 27. A high affinity monoclonal antibody product binder is linked to a substance that non-covalently binds to the vitreous. The high affinity monoclonal antibody product binder linked to the substance that non-covalently binds the vitreous is inserted into the vitreous cavity. Monoclonal antibody products that are subsequently inserted into the vitreous cavity are retained in the vitreous cavity through binding to the high affinity monoclonal antibody product binder that is anchored by noncovalent binding of the substance to the vitreous thereby increasing the resident time and duration of action of monoclonal antibody products in the eye.

FIG. 28A-28F. In situ modeling of the effect of an anchored high affinity monoclonal antibody product binder on the duration of Bevacizumab (Avastin) injected into the vitreous cavity of the eye.

FIG. 29. In vitro survival of ARPE-19 cells exposed to protein A.

FIG. 30. Histopathologic Results.

FIG. 31. Enzymatic cleavage sites. Depicted is SpGc3 (SEQ ID NO:4); protein A Z-domain (SEQ ID NO:3); Protein A minimized Z domain (SEQ ID NO:1) and cysteine stabilized Z domain (SEQ ID NO:2), each of which provides high affinity binding to a therapeutic antibody Fc region. Each can be independently joined to an anchoring domain, exemplified here as LINK (SEQ ID NO:22); or hyaluronic acid binding peptides (SEQ ID NO:23, 24, 25, 26, 27, respectively).

FIG. 32. Peptide based high affinity monoclonal antibody product binders anchored in the vitreous cavity with hyaluronic acid peptide binder.

FIG. 33. Synthetic scheme for 2,4,6 Trisubstituted Triazine.

FIG. 34. Non-peptide based high affinity monoclonal antibody binders with non-peptide anchoring in the vitreous cavity.

FIG. 35. Non-peptide based high affinity monoclonal antibody binders with non-peptide based anchoring in the vitreous cavity.

FIG. 36. Non-peptide based high affinity monoclonal antibody binders with peptide based anchoring in the vitreous cavity.

FIG. 37. Non-peptide based high affinity monoclonal antibody binders with polymer based anchoring in the vitreous cavity.

FIG. 38. Peptide based high affinity monoclonal antibody binders with polymer method of anchoring in the vitreous cavity.

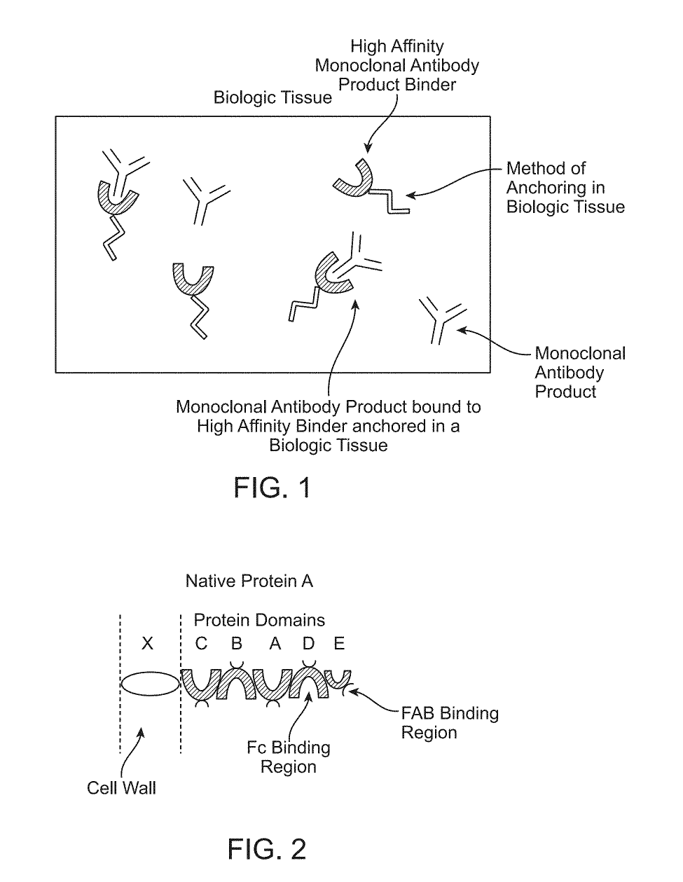

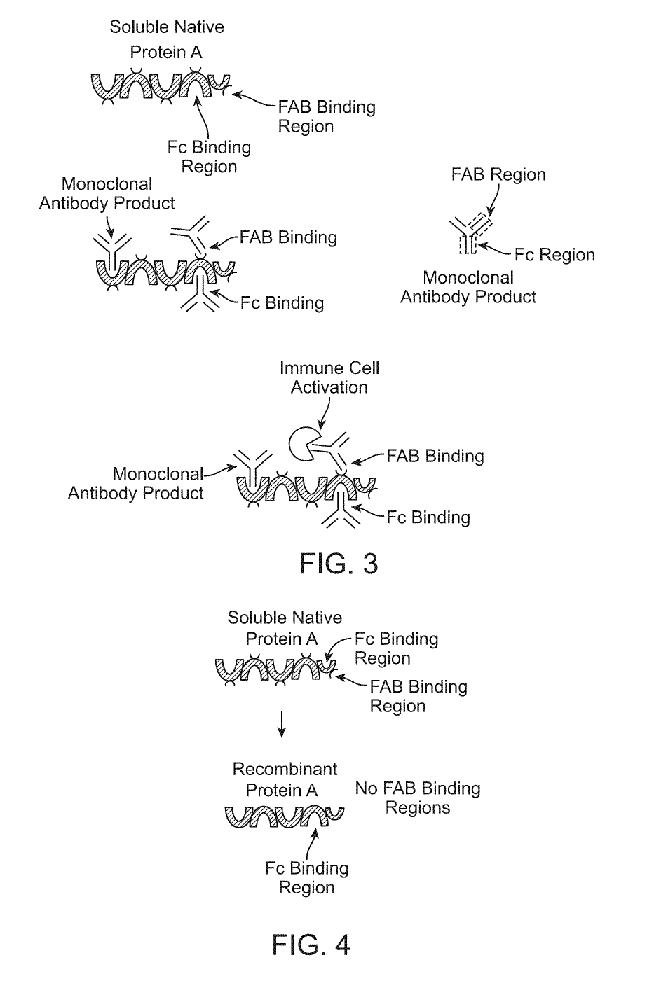

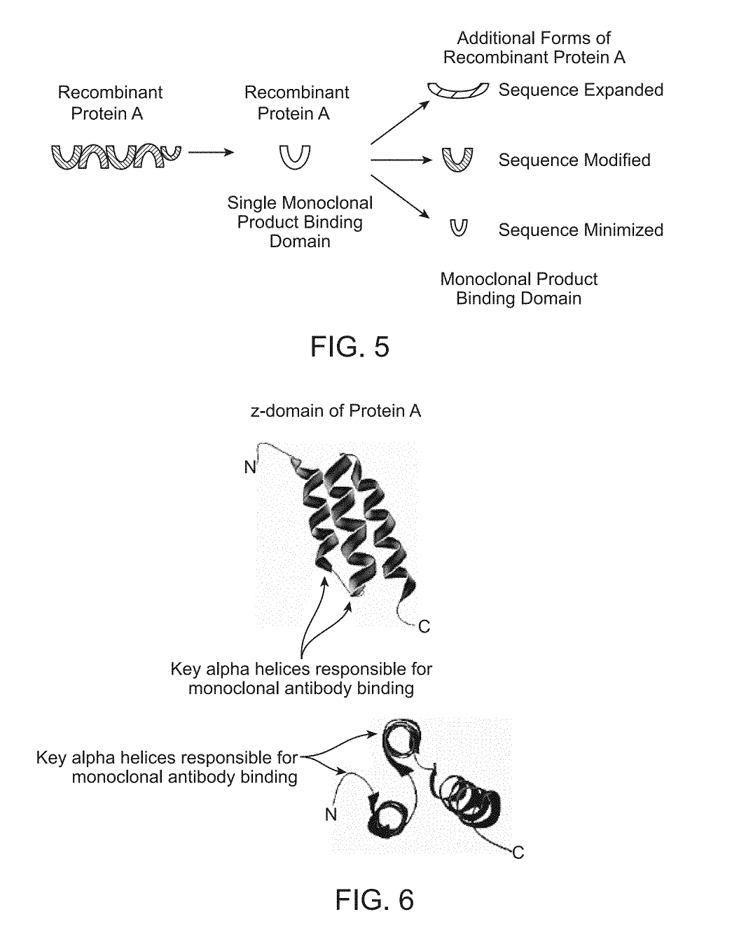

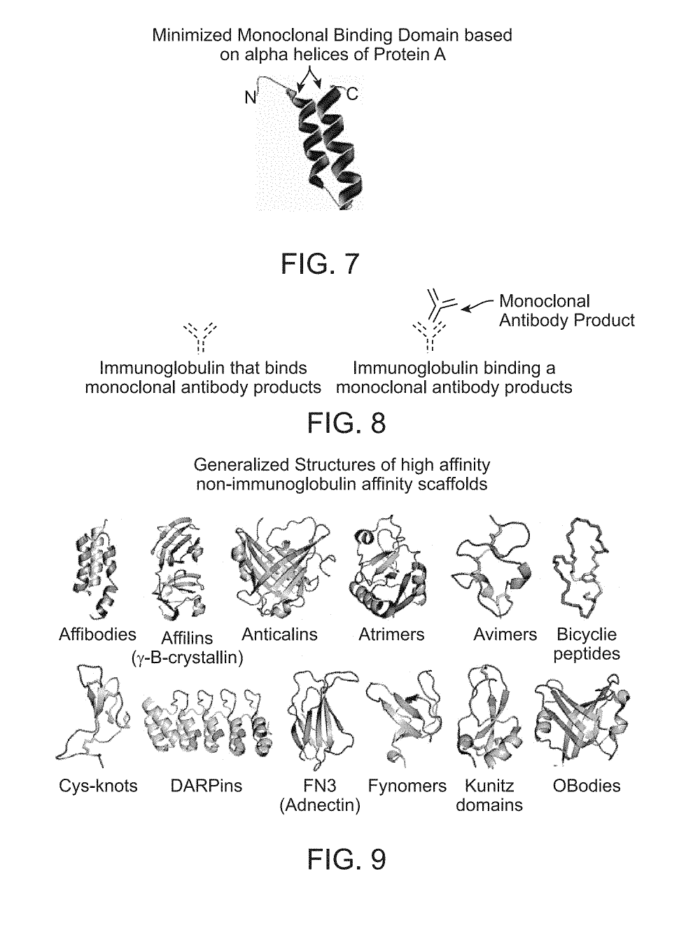

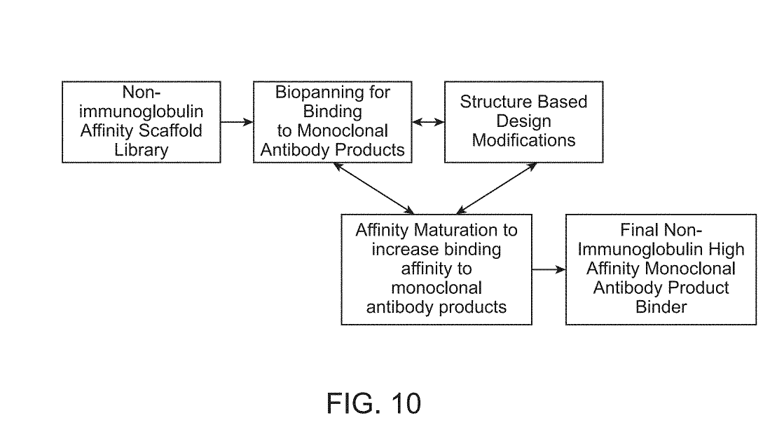

### DETAILED DESCRIPTION

Provided herein is a description of molecules useful in combination therapy with therapeutic antibodies, where retention in a specific tissue of interest is desirable. The molecules have the property of (1) binding monoclonal antibody products with a dissociation constant (K_{D}) less than about 10⁻⁶ M K_{D}, less than about 10⁻⁷ M K_{D}, less than about 10⁻⁸ M less than about 10⁻⁶ M K_{D}, or less. In some embodiments the binding affinity is less than about 10⁻⁷ M K_{D}; and (2) anchoring in a biologic tissue of interest. These two components are the key defining features. There are a number of different substances disclosed herein; and methods of anchoring each of these substances to a biologic tissue. What follows is an overview of different embodiments. Those skilled in the art are capable of making obvious modifications to the various substances and methods of anchoring in a biologic tissue that are disclosed in what follows. Though not expressly stated, such modifications are included within the scope of the present invention.

### Definitions

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

“In combination with”, “combination therapy” and “combination products” refer, in certain embodiments, to the concurrent administration to a patient of the agents described herein. i.e. an anchor and a therapeutic antibody. A therapeutic antibody may be defined as an antibody that accomplishes a change in the condition for treatment, which activity is enhanced by a combination therapy with the anchor. When administered in combination, each component can be administered at the same time or sequentially in any order at different points in time. Thus, each component can be administered separately but sufficiently closely in time so as to provide the desired therapeutic effect.

“Concomitant administration” of active agents in the methods of the invention means administration with the reagents at such time that the agents will have a therapeutic effect at the same time. Such concomitant administration may involve concurrent (i.e. at the same time), prior, or subsequent administration of the agents. A person of ordinary skill in the art would have no difficulty determining the appropriate timing, sequence and dosages of administration for particular drugs and compositions of the present invention.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acid modifications disclosed herein may include amino acid substitutions, deletions and insertions. Variant proteins may also include conservative modifications and substitutions at other positions of the receptor (e.g. positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: ala, pro, gly, gin, asn, ser, thr; Group II: cys, ser, tyr, thr; Group III: val, ile, leu, met, ala, phe; Group IV: lys, arg, his; Group V: phe, tyr, trp, his; and Group VI: asp, glu. Further, amino acid substitutions with a designated amino acid may be replaced with a conservative change.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been intentionally modified.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having disease. Subjects may be human, but also include other mammals, particularly those mammals useful as laboratory models for human disease, e.g. mouse, rat, etc.

The term “diagnosis” is used herein to refer to the identification of a molecular or pathological state, disease or condition.

The term “prognosis” is used herein to refer to the prediction of the likelihood of death or progression, including recurrence, spread, and drug resistance. The term “prediction” is used herein to refer to the act of foretelling or estimating, based on observation, experience, or scientific reasoning. In one example, a physician may predict the likelihood that a patient will survive.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of AMD, or cancer, etc. in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

As used herein, a “therapeutically effective amount” refers to that amount of the therapeutic agent, e.g. therapeutic antibody administered in combination with an anchor, sufficient to treat or manage a disease or disorder. A therapeutically effective amount may refer to the amount of therapeutic agent sufficient to delay or minimize the onset of disease, e.g., delay or minimize the spread of cancer, or the amount treat AMD, etc. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the terms “prevent”, “preventing” and “prevention” refer to the prevention of the recurrence or onset of one or more symptoms of a disorder in a subject as result of the administration of a prophylactic or therapeutic agent.

Dosing Regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

The term “antibody” herein is used in the broadest sense and specifically covers monoclonal antibodies, polyclonal antibodies, monomers, dimers, multimers, multispecific antibodies (e.g., bispecific antibodies), heavy chain only antibodies, three chain antibodies, single chain Fv, nanobodies, etc., and also include antibody fragments, so long as they exhibit the desired biological activity (Miller et al (2003) Jour. of Immunology 170:4854-4861). Antibodies may be murine, human, humanized, chimeric, or derived from other species. Of particular interest is any therapeutic protein comprising an IgG Fc region sequence, for example a VEGF trap fused to an Fc region; monoclonal antibodies, bispecific antibodies, heavy chain only antibodies, and the like.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2); usually an IgG class or subclass of immunoglobulin molecule, including engineered subclasses with altered Fc portions that provide for reduced or enhanced effector cell activity. The immunoglobulins can be derived from any species. In one aspect, the immunoglobulin is of largely human origin.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

An “intact antibody chain” as used herein is one comprising a full length variable region and a full length constant region. An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or

IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called K and A, based on the amino acid sequences of their constant domains.

A “functional Fc region” possesses an “effector function” of a native-sequence Fc region, and can bind with high affinity to an anchor molecule. A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof. The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, an antibody having an Fc region according to this invention can comprise an antibody with or without K447.

“Native antibodies and immunoglobulins” are typically heterotetrameric glycoproteins of about 150,000 daltons, composed of two light (L) chains and two heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains, in the configuration V_{H}-C_{H1}-H-C_{H2}-C_{H3}. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (see, for example, Clothia et al., J. Mol. Biol. 186:651 (1985); Novotny and Haber, Proc. Natl. Acad. Sci. U.S.A. 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Immunoglobulin G (IgG) is one of the most abundant proteins in human serum, accounting for about 10-20% of plasma protein. It is the major class of the five classes of immunoglobulins in human beings, IgM, IgD, IgG, IgA, and IgE. IgG can be further divided in four subclasses, named, in order of decreasing abundance IgG1, IgG2, IgG3, and IgG4. Although they are more than 90% identical on the amino acid level, each subclass has a unique profile with respect to antigen binding, immune complex formation, complement activation, triggering of effector cells, half-life, and placental transport.

“Specific binding moiety”, as used herein, refers to an agent, for example a polypeptide or a triazine compound, that interacts specifically to associate with a binding partner of interest where the relative binding constant (Kd) is sufficiently strong to allow, for example, detection of binding to the protein by a detection means; physiologically relevant association, etc. In some embodiments, the affinity of one molecule for another molecule to which it specifically binds is characterized by a K_{D} (dissociation constant) of 10⁻⁶ M or less (e.g., 10⁻⁷ M or less, 10⁻⁶ M or less, 10⁻⁹ M or less, 10⁻¹⁰ M or less, 10⁻¹¹ M or less, 10⁻¹² M or less, 10⁻¹³ M or less, 10⁻¹⁴ M or less, or 10⁻¹⁵ M or less). “Affinity” refers to the strength of binding, increased binding affinity being correlated with a lower dissocation constant (K_{D}).

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member). Suitable specific binding members include agents that specifically bind antibodies; that specifically bind components of biological tissues such as hyaluronic acid, etc. Other specific binding moieties of interest include, for example, ligand/receptor, or receptor and counter-receptor binding partners, where the binding moiety may be a native binding partner, an affinity matured binding partner, etc.

“Binding affinity” generally refers to the strength of the sum total of noncovalent interactions between a single binding site of a molecule (e.g., an antibody or other binding molecule) and its binding partner (e.g., an antigen or receptor). The affinity of a molecule X for its partner Y can generally be represented by the dissociation constant (Kd), where lower dissociation constants are indicative of stronger binding. Affinity can be measured by common methods known in the art, including those described herein. Low-affinity antibodies bind antigen (or receptor) weakly and tend to dissociate readily, whereas high-affinity antibodies bind antigen (or receptor) more tightly and remain bound longer.

The binding properties of a binding agent, e.g. the affinity between an Fc region and a candidate anchor molecule; or between a biological tissue component such a hyaluronic acid and a candidate binding molecule, may be measured by any method, e.g., one of the following methods: BIACORE™ analysis, Enzyme Linked Immunosorbent Assay (ELISA), x-ray crystallography, sequence analysis and scanning mutagenesis. The binding interactions can be analyzed using surface plasmon resonance (SPR). SPR or Biomolecular Interaction Analysis (BIA), which detects biospecific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (Kd), and kinetic parameters, including Kon and Koff, for the binding of a molecule to a target. Such data can be used to compare different molecules. Information from SPR can also be used to develop structure-activity relationships (SAR). For example, the kinetic and equilibrium binding parameters of different molecule can be evaluated. Variant amino acids at given positions can be identified that correlate with particular binding parameters, e.g., high affinity and slow Koff. This information can be combined with structural modeling (e.g., using homology modeling, energy minimization, or structure determination by x-ray crystallography or NMR).

Age-Related Macular Degeneration (AMD or ARMD) is a condition of interest for treatment with a combination therapy described herein. Age-related macular degeneration (AMD) is the most common cause of irreversible central vision loss in elderly patients. Dilated funduscopic findings are diagnostic; color photographs, fluorescein angiography, and optical coherence tomography assist in confirming the diagnosis and in directing treatment. Treatment can include intravitreal injection of antivascular endothelial growth factor drugs.

Two different forms of AMD occur. Dry (nonexudative or atrophic): All AMD starts as the dry form. About 85% of people with AMD have only dry AMD. Wet (exudative or neovascular) AMD occurs in about 15% of people. Although only 15% of patients with AMD have the wet form, 80 to 90% of the severe vision loss caused by AMD results from wet AMD.

Dry AMD causes changes of the retinal pigment epithelium, typically visible as dark pinpoint areas. The retinal pigment epithelium plays a critical role in keeping the cones and rods healthy and functioning well. Accumulation of waste products from the rods and cones can result in drusen, which appear as yellow spots. Areas of chorioretinal atrophy (referred to as geographic atrophy) occur in more advanced cases of dry AMD. There is no elevated macular scar (disciform scar), edema, hemorrhage, or exudation. The loss of central vision occurs over years and is painless, and most patients retain enough vision to read and drive. Central blind spots (scotomas) usually occur late in the disease and can sometimes become severe. Symptoms are usually bilateral. Funduscopic changes include the following: Changes in the retinal pigment epithelium, Drusen, areas of chorioretinal atrophy.

Wet AMD occurs when new abnormal blood vessels develop under the retina in a process called choroidal neovascularization (abnormal new vessel formation). Localized macular edema or hemorrhage may elevate an area of the macula or cause a localized retinal pigment epithelial detachment. Eventually, untreated neovascularization causes a disciform scar under the macula. Rapid vision loss, usually over days to weeks, is more typical of wet AMD. The first symptom is usually visual distortion, such as a central blind spot (scotoma) or curving of straight lines (metamorphopsia). Peripheral vision and color vision are generally unaffected; however, the patient may become legally blind (<20/200 vision) in the affected eye, particularly if AMD is not treated. Wet AMD usually affects one eye at a time; thus, symptoms of wet AMD are often unilateral. Funduscopic changes include the following: Subretinal fluid, appearing as localized retinal elevation, Retinal edema, Gray-green discoloration under the macula, Exudates in or around the macula, Detachment of retinal pigment epithelium (visible as an area of retinal elevation), subretinal hemorrhage in or around the macula.

Both forms of AMD are diagnosed by funduscopic examination. Visual changes can often be detected with an Amsler grid. Color photography and fluorescein angiography are done when findings suggest wet AMD. Angiography shows and characterizes subretinal choroidal neovascular membranes and can delineate areas of geographic atrophy. Optical coherence tomography (OCT) aids in identifying intraretinal and subretinal fluid and can help assess response to treatment.

Antibody-based treatment of late-stage neovascular AMD with inhibitors of vascular endothelial growth factor has had great success, which is now the goal for currently untreatable AMD manifestations. The existence of an immune-privileged environment in the eye supports the feasibility of localized antibody therapy, which benefit from extended duration by a combination therapy with an anchor as described herein. Many different antibodies against various targets are being developed for the treatment of AMD, which reflects the etiological complexity of the disease.

Targets for therapeutic antibodies include inhibition of angiogenic proteins production; angiogenic protein neutralization; angiogenic proteins receptors inhibition or endothelial cell apoptosis induction. Almost every ophthalmic drug either currently available or under development for the treatment of AMD began life as a cancer therapy. For example ranibizumab (Lucentis®) and bevacizumab (Avastin®) are currently in clinical use for wet AMD. Aflibercept, (Eylea®), results of a process of bioengineering where extramembrane fragments of receptors 1 and 2 of VEGF are merged to IgG1 FC fragment. This recombinant fusion protein is a composite decoy receptor based on VEGF receptors VEGFR1 and VEGFR2. The composition as therapeutic antibody products and the rapid clearance of Avastin®, Lucentis® and Eylea® from the vitreous cavity all allow them to benefit from combination therapy with an anchor. Volociximab (Ophthotech) is a high-affinity chimeric monoclonal antibody (Mab) that inhibits the functional activity of alpha-5 beta-1 integrin found on the endothelial cells involved in the formation of blood vessels. Eculizumab (Soliris, Alexion Pharmaceuticals) is a humanized monoclonal antibody derived from a murine antihuman C5 antibody. Eculizumab specifically inhibits the terminal complement protein C5, thereby preventing its cleavage to C5a and C5b during complement activation. RG7715 a humanized monoclonal antibody with bispecific Ang2 inhibitor/anti-VEGF biologic. ICON-1 is an immunoconjungate containing an IgG1 Fc receptor and a binding domain for Tissue Factor. In all of these cases, these antibody therapeutics benefit from increased duration of action through combination therapy with an anchor.

Diabetic retinopathy (DR), which includes both proliferative (PDR) and non-proliferative (NPDR) and diabetic macular edema (DME) (including focal, non-center DME and diffuse, center-involved DME), is a condition of interest for treatment with a combination therapy described herein. Diabetic retinopathy is a consequence of impaired glucose regulation that affects over 100 million people worldwide. Impaired glucose metabolism cause damage to capillary beds in the retina resulting in local ischemia. This ischemia then causes pathologic production of a number of different compounds, most notably vascular endothelial growth factor (VEGF). Elevation in VEGF causes macular edema (DME) and abnormal vessel proliferation (PDR) that can lead to vitreous hemorrhage or tractional retinal detachments.

Antibody-based treatment diabetic retinopathy includes many of the same therapeutics used to treat AMD, including Avastin®, Lucentis®, and Eylea®. Newer therapeutic monoclonal antibody products are also in development reflecting the etiological complexity of the disease. In each of these cases, the need to the duration of action of these therapeutic antibody products can be achieved by a combination therapy with an anchor as described herein.

Because of the therapeutic efficacy of therapeutic antibodies there are a number of other ocular diseases in which treatment with these antibodies has been used successfully including, for example, ischemia-related retinopathies, retinal vein occlusion (RVO) (including central (CRVO) and branched (BRVO) forms), choroidal neovascularization (CNV) (including myopic CNV, histoplasmosis associated CNV, and posterior uveitis associated CNV), retinal neovascularization, von Hippel-Lindau disease (VHL), retinopathy of prematurity (ROP), familial exudative vitreoretinopathy (FEVR), Coats' disease, Norrie Disease, neovascularization of the iris or angle, hypertensive retinopathy, retinal angiomatous proliferation, macular telangiectasia, cystoid macular edema (CME), uveitis (including infectious and non-infectious uveitis), vasculitis, retinitis, and choroiditis. In each of these conditions, the existence of an immune-privileged environment in the eye supports the feasibility of localized antibody therapy, which benefit from extended duration by a combination therapy with an anchor as described herein.

Treatment of arthropathies with intra-articular therapeutic antibodies is another area that benefits from increased duration of action through combination therapy with an anchor as described herein. A number of arthropathies are currently treated with systemic administration of monoclonal antibody products including: adult-onset still's disease, ankylosing spondylitis, Bechet's disease, bursitis, calcium pyrophosphate deposition disease (CPPD) carpal tunnel syndrome, degenerative, degenerative disc disease, familial mediterranean fever, fibromyalgia, fifth disease, giant cell arteritis, gout, hemochromatosis, inflammatory arthritis, inflammatory bowel disease, juvenile arthritis, juvenile dermatomyositis (JD), juvenile idiopathic arthritis (JIA), juvenile scleroderma, Kawasaki disease, Lyme disease, mixed connective tissue disease, myositis (including polymositis, dermatomyositis), osteoarthritis, pagets, palindromic rheumatism, patellofemoral pain syndrome, pediatric rheumatic diseases, pediatric SLE, polymyalgia rheumatic, pseudogout, psoriatic arthritis, Raynaud's phenomenon, reactive arthritis, reflex sympathetic dystrophy, reiter's syndrome, rheumatic fever, rheumatoid arthritis, scleroderma, Sjogren's disease, spinal stenosis, spondyloarthritis, systemic juvenile idiopathic arthritis, systemic lupus erythematosus (SLE), granulomatosis with polyangiitis, vasculitis.

Despite improvements in these conditions, systemic administration of these monoclonal antibody products has a number of systemic adverse effects, most notably severe, life threatening infection. For some of the aforementioned diseases, arthritis localized to one or more joints may be the primary complaint. In these instances, there is tremendous interest in intra-articular delivery of therapeutic monoclonal antibody products to treat the disease process locally and avoid systemic adverse events. Currently, over 16 clinical trials are examining whether intra-articular monoclonal antibody products are effective are underway. Unfortunately, due to lymphatic drainage around the joint space, monoclonal antibody products are rapidly cleared from the synovium via lymphatics with monoclonal antibody half-lives measured in hours. The result is that many of these trials of intra-articular administration has been disappointing.

There are a number of systemically administered therapeutic antibodies that would have significant efficacy as intra-articular injections if retention of these antibodies in the joint space could be increased. Examples of such antibodies include: tissue necrosis factor (TNF) inhibitors which are composed of monoclonal antibodies that have IgG1 Fc regions include infliximab, adalimumab, golimumab, entanercept, and certolizumab; monoclonal antibodies targeting B cells such as rituximab; monoclonal antibodies targeting IL-6 such as tocilizumab, siltuximab, olokizumab, elisilimomab, clazakizumab, and sirukumab; monoclonal antibodies targeting IL-1 including canakinumab. All of these antibodies can be administered intra-articularly and benefit from extended duration by a combination therapy with an anchor as described herein.

Treatment of cancer with a therapeutic antibody can benefit from methods of retaining the therapeutic antibody in targeted tissue with the methods provided herein. The term “cancer” (or “cancerous”), “hyperproliferative,” and “neoplastic” to refer to cells having the capacity for autonomous growth (e.g., an abnormal state or condition characterized by rapidly proliferating cell growth). Hyperproliferative and neoplastic disease states may be categorized as pathologic (e.g., characterizing or constituting a disease state), or they may be categorized as non-pathologic (e.g., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

Examples of tumor cells include but are not limited to AML, ALL, CML, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

A number of antibodies that target tumor cell antigens are currently in clinical use for the treatment of cancer, and others are in varying stages of clinical development. For example, there are a number of antigens and corresponding monoclonal antibodies for the treatment of B cell malignancies. One target antigen is CD20. Rituximab is a chimeric unconjugated monoclonal antibody directed at the CD20 antigen. CD20 has an important functional role in B cell activation, proliferation, and differentiation. The CD52 antigen is targeted by the monoclonal antibody alemtuzumab, which is indicated for treatment of chronic lymphocytic leukemia. CD22 is targeted by a number of antibodies, and has recently demonstrated efficacy combined with toxin in chemotherapy-resistant hairy cell leukemia. Two new monoclonal antibodies targeting CD20, tositumomab and ibritumomab, have been submitted to the Food and Drug Administration (FDA). These antibodies are conjugated with radioisotopes. Alemtuzumab (Campath) is used in the treatment of chronic lymphocytic leukemia; Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer.

The CD52 antigen is targeted by the monoclonal antibody alemtuzumab, which is indicated for treatment of chronic lymphocytic leukemia; colon cancer and lung cancer. CD22 is targeted by a number of antibodies, and has recently demonstrated efficacy combined with toxin in chemotherapy-resistant hairy cell leukemia.

Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer.

Cetuximab (Erbitux) is also of interest for use in the methods. The antibody binds to the EGF receptor (EGFR), and has been used in the treatment of solid tumors including colon cancer and squamous cell carcinoma of the head and neck (SCCHN).

Monoclonal antibodies useful in the methods of the invention that have been used in solid tumors include, without limitation, edrecolomab and trastuzumab (herceptin). Edrecolomab targets the 17-1A antigen seen in colon and rectal cancer, and has been approved for use in Europe for these indications. Trastuzumab targets the HER-2/neu antigen. This antigen is seen on 25% to 35% of breast cancers. Cetuximab (Erbitux) is also of interest for use in the methods of the invention. The antibody binds to the EGF receptor (EGFR), and has been used in the treatment of solid tumors including colon cancer and squamous cell carcinoma of the head and neck (SCCHN).

### Compositions

Anchor molecules, which are useful in combination therapies with therapeutic antibodies and other therapeutic agents comprising an antibody Fc region sequence, comprise a region or moiety that binds to an antibody, usually a region or moiety that binds to a human IgG Fc region sequence, at high affinity with a dissociation constant (K_{D}) that may be less than about 10⁻⁶ M, less than about 10⁻⁷ M, less than about 10⁻⁶ M, less than about 10⁻⁹ M, or less. The region or moiety that binds to an antibody is linked, fused, covalently joined, or conjugated to a region or moiety that provides for anchoring in a biological tissue of interest.

### Antibody Binding Region or Moiety

High affinity binding regions or moieties can be classified as: proteins and peptides based on naturally occurring binders, proteins or peptides based on non-immunoglobulin affinity scaffolds, immunoglobulins or fragments thereof, and non-peptide based substances, which bind with high affinity to therapeutic antibodies, e.g. IgG Fc regions.

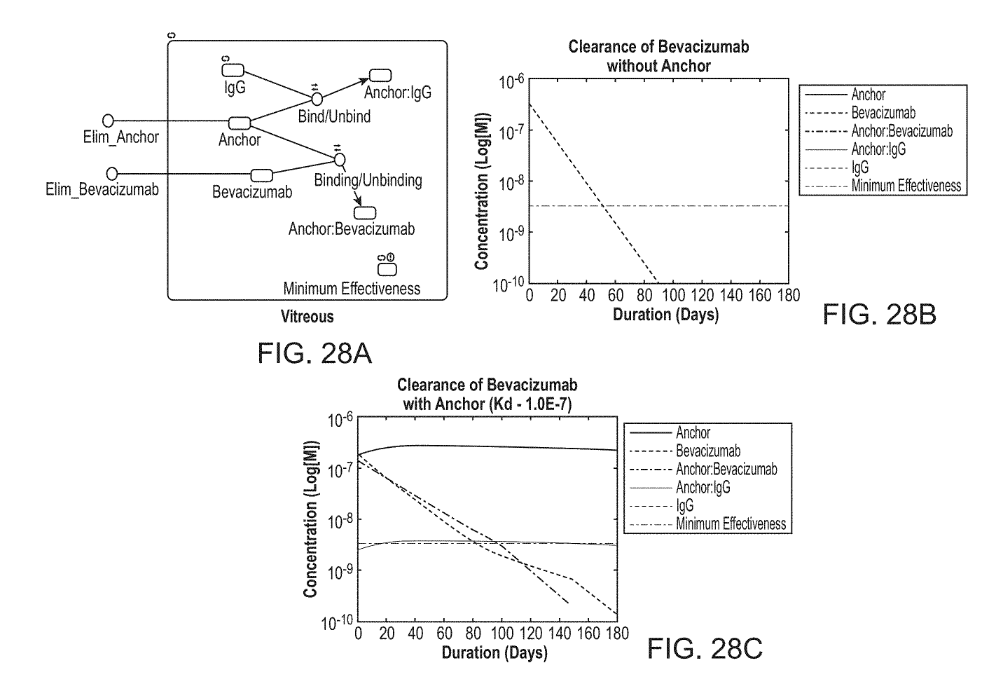

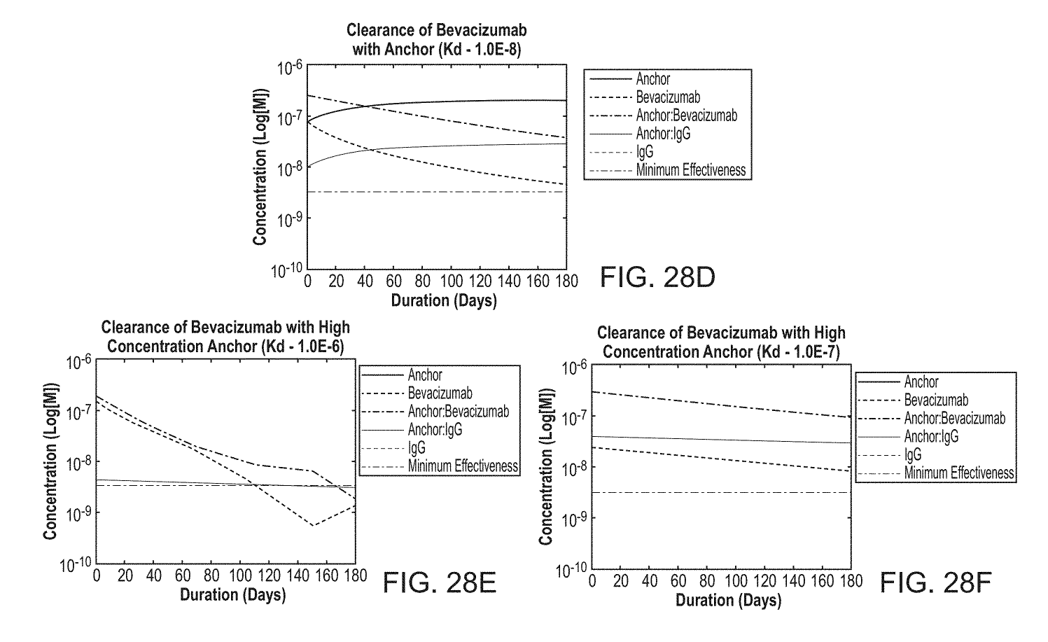

In order to retain therapeutic monoclonal antibodies in a biologic tissue, high affinity binders must have an affinity with a dissociation constant (K_{D}) of less than 10⁻⁶ M. The importance of this high affinity binding has been demonstrated with in silico modeling of bevacizumab diffusion from the eye using a two-compartment model (FIG. 28). Based on these requirements, a number of peptide and non-peptide variants have been identified and tested.

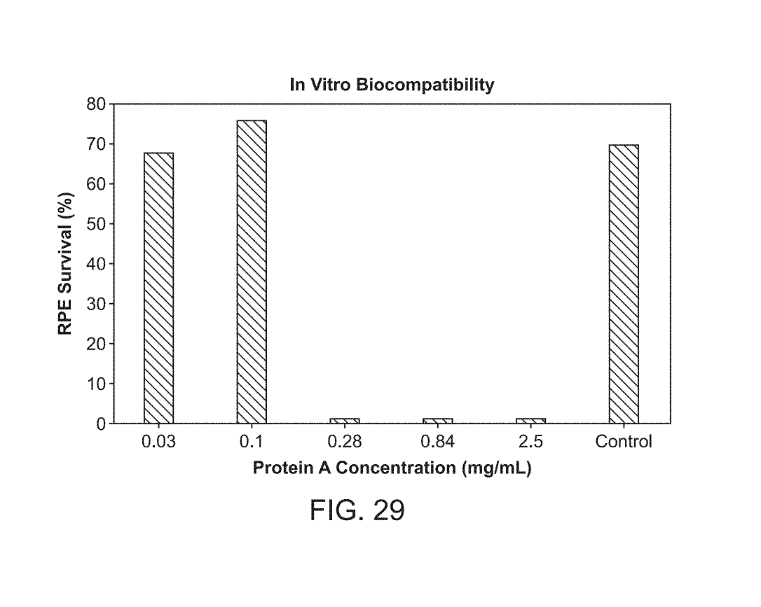

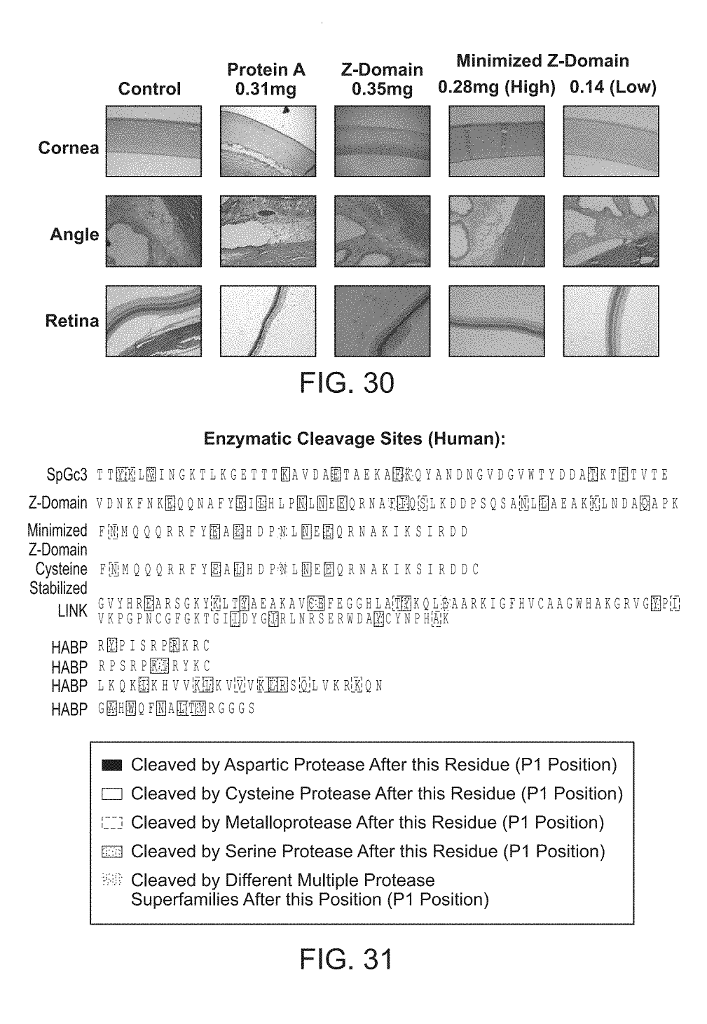

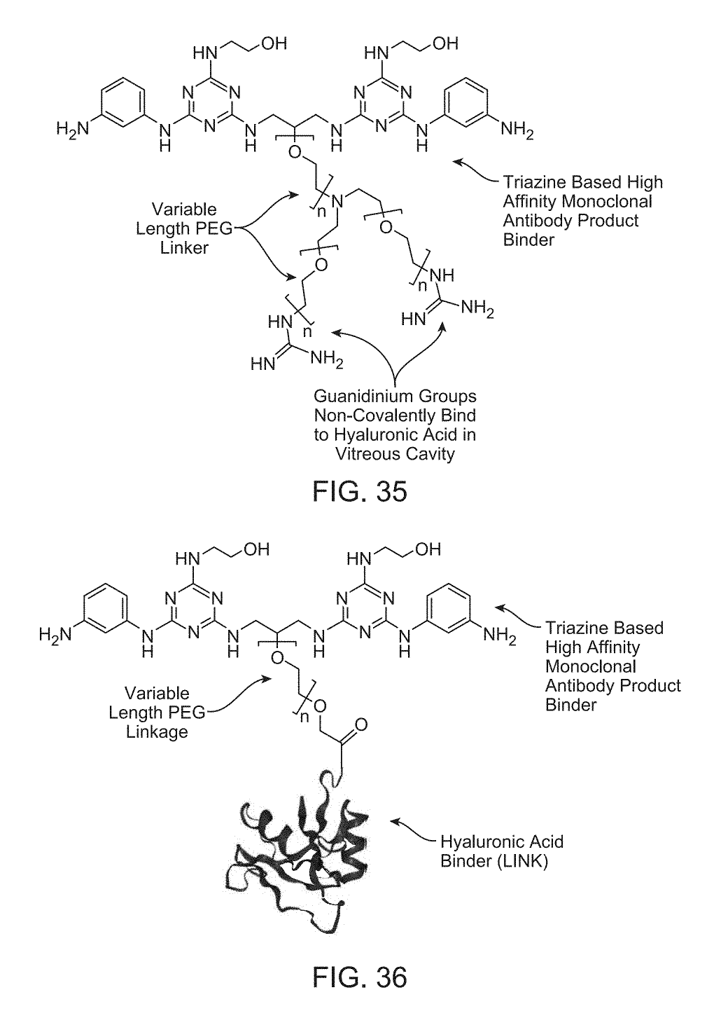

Exemplary examples of high affinity binders are the listed in Table 1, both peptide variants (SEQ ID NO: 1-14) and non-peptide variants. Both the peptide based and non-peptide based high affinity binders are ideal candidates because they are easily chemically synthesized, have high binding affinity for monoclonal antibody products, and can be readily bound to anchoring moieties. Of particular interest are SEQ ID NO: 1 (and its sister sequence, SEQ ID NO: 2). These sequences are minimized versions of the Z-domain, have dissociation constants (K_{D}) less than 10⁻⁷M, and have shown both in vitro and in vivo biocompatibility (FIGS. 29 and 30). The non-peptide based variants are also of particular interest. Both of these non-peptide high affinity binders are triazine-based compounds which mimic the binding pocket of protein A. They are both easily synthesized (FIG. 33), have high binding affinities with dissociation constants (K_{D}) of around 10⁻⁷M, and are well tolerated in vivo, with a wide therapeutic index (Zacharie et al. (2010) Journal of medicinal chemistry 53.3: 1138-1145) making them well suited for in vivo use to bind therapeutic monoclonal antibodies. The presence of a free hydroxyl group on linker sequence between triazine rings on compound 1 provides excellent location for conjugation to an anchoring moiety (discussed in detail below) and is demonstrated in FIG. 35.

Optimization of these high affinity binders listed in Table 1 for in vivo use can involve changes made to resist proteolytic or chemical degradation within the biologic tissue. Potential enzymatic cleavage sites of a subset of sequences listed in Table 1 are highlighted in FIG. 31. Modifications to resist proteolytic or chemical degradation include, but are not limited to, substitution at the nitrogen atom to create protease-resistant peptides (peptoids), incorporation of alpha-methyl functionalized amino acids directly into the main chain during standard protein synthesis, insertion of or substitution with non-proteinogenic (“unnatural”) animo acids, inclusion of or substitution with non-natural beta-amino acids, inclusion of or substitution with non-natural beta-amino acids specifically before or after enzymatic cleavage sites as predicted by protease prediction software, substitution of alpha amino acids in an alpha helix with beta amino acids in an ααβ, αααβ, ααβαααβ, or αₙβₘ configuration, or unordered substitution of alpha amino acids with beta amino acids that maintains the secondary or tertiary structure and/or binding affinity, inclusion of or substitution with non-natural d-amino acids, inclusion of or substitution with d-amino acids at the terminal ends of a peptide sequence, full or partial sequence reversal with d-amino acids substitutions (known as retro-inverso peptides) or other non-natural amino acid substitutions in order to maintain the secondary or teritiary structure and/or binding, addition of hyaluronic acid, polyethylene glycol, or other hydrogel moieties. Combinations of one or more of the aforementioned modifications are envisioned. In particular, α/β amino acid substitutions of the alpha helices of SEQ ID NO: 1 and 2, substitution of the enzymatic cleavage P1′ amino acids with non-natural amino acids, and addition of d-peptides both at the N- and C-terminus and as partial retro-inverso sequences are envisioned to prevent in vivo proteolysis and/or immunologic recognition.

TABLE 1

| SEQ ID | | |
|---|---|---|
| 1 | FNMQQQRRFYEALHDPNLNEEQRNAKIKSIRDD | Minimized Z-Domain |
| 2 | FNMQCQRRFYEALHDPNLNEEQRNAKIKSIRDDC | Cysteine Stabilized |
| | | Sequence |
| 3 | VDNKFNKEQQNAFYEILHLPNLNEEQRNAFIQSLKDDPSQS | Z domain |
| | ANLLAEAKKLNDAQAPK | |
| 4 | TTYKLVINGKTLKGETTTKAVDAETAEKAFKQYANDNGVDG | SpGc3 |
| | VWTYDDATKTFTVTE | |
| 5 | SVKVKFXXXGEEKEVDTSKIRDVCRQGKNVKFLYNDNGKYG | Sac7d |
| | AGXVXEKDAPKELLDMLARAEREKK | |
| 6 | EPIHRSTLTALL | short peptide |
| 7 | FYWHCLDE | short peptide |
| 8 | HWRGWV | hexamer |
| 10 | (CFFHH)₂KG | Peptide H |
| 11 | (RTY)₄(K)₂KG | Tetrameric |
| | | Tripeptide (PAM) |
| 12 | (dRdTdY)₄(K)₂KG | Tetrameric |
| | | Tripeptide Partial |
| | | Inverso (D-PAM) |
| 13 | (GFRKYLHFRRHLL)₄(K)₂KG | Tetrameric |
| | | Tripeptide |
| 14 | (VRLGWLLAPADLDAR)₄(KRG)₂KG | Tetrameric |
| | | Tripeptide |
| | 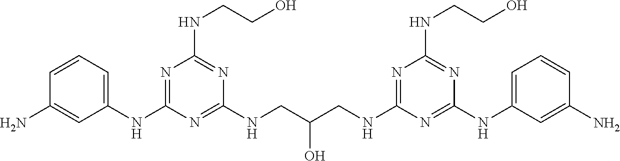 [SMILES: Nc1cccc(Nc2nc(NCCO)nc(NCC(O)CNc3nc(NCCO)nc(Nc4cccc(N)c4)n3)n2)c1] | Compound 1 1,3-bis({4-[(3- aminophenyl)amino]- 6-[(2- hydroxyethyl)amino]- 1,3,5-triazin-2- yl}amnio)propan-2-ol |
| | 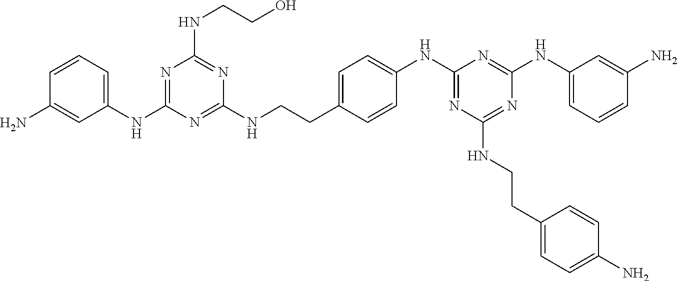 [SMILES: Nc1ccc(CCNc2nc(Nc3ccc(CCNc4nc(NCCO)nc(Nc5cccc(N)c5)n4)cc3)nc(Nc3cccc(N)c3)n2)cc1] | Compound 2 2-({4-[(3- aminophenyl)amino]- 6-({2-[4-({4-[(3- aminophenyl)amino]- 6-{[2-(4- aminophenyl)ethyl] amino}-1,3,5- triazin-2- yl}amino)phenyl] ethyl}amino)-1,3,5- triazin-2- yl}amino)ethan-1-ol |

Additional high affinity binders of antibodies based on naturally occurring binding proteins include those based on, but not limited to, gp120, Protein A (SpA), Protein G, Protein NG, Protein L (PpL), Staphylococcal enterotoxin D (SED), P. falciparum erythrocyte membrane protein 1 (PfEMP1), and Protein Fv (pFv), and Fc receptors (e.g. Fcγ). Recombinant forms of these naturally occurring binders include, without limitation, minimized, truncated, modified, or elongated forms, single or multiple changes in the amino acid sequence, or any other modifications to the primary, secondary, or tertiary structure for the purpose of enhancing binding, reducing immunogenicity, resisting degradation, and/or eliminating off target-binding.

Additionally, polypeptides based entirely on or in part on these naturally occurring high affinity binders are used. For instance, the Z-domain (SEQ ID NO: 3) is a synthetic hybrid domain based on the five binding domains of Protein A. Further modifications to this Z-domain such as minimization, truncation, fusion, elongation, or sequence or structure modification to reduce or eliminate off-target binding are also of interest. For instance, SEQ ID NO:1 and 2 are minimized versions of the Z-domain (see, for example, U.S. Pat. No. 6,013,763, herein specifically incorporated by reference for peptide variants described therein). Short peptides derived from the binding domains of these naturally occurring proteins such as, but not limited to, branched peptides such as Peptide H (CFFHH)₂-K-G (SEQ ID NO:10), PAM ((RTY)₄-K2-K-G) (SEQ ID NO:11), TG19318 ((rty)₄-K2-K-G) (SEQ ID NO: 12), (GFRKYLHFRRHLL)₄(K)₂KG (SEQ ID NO: 13), (VRLGWLLAPADLDAR)₄(KRG)₂KG (SEQ ID NO: 14) and/or linear peptides like EPIHRSTLTALL (SEQ ID NO: 6), FYWHCLDE (SEQ ID NO: 7) HWRGWV (SEQ ID NO:8) are also envisioned in this disclosure. The peptide SpGc3 (SEQ ID NO: 4) is an example of a peptide derived from protein G. Examples of high affinity binding polypeptides may be found, for example, in U.S. Pat. No. 6,013,763A (Braisted, 1996); WO2016031909A1 (Yoshida, 2016); U.S. Pat. No. 5,831,012A (Nilsson, 1995); Ehrlich and Bailon Journal of Biochemical and Biophysical Methods 49.1 (2001): 443-454; US20060153834A1 (Carbonell, 2006); and U.S. Pat. No. 6,207,807B1 (Fassina, 1995); each herein specifically incorporated by reference.

Other high affinity binders that bind monoclonal antibody products and are based on non-immunoglobulin affinity scaffolds include, but are not limited to, domain-sized compounds and constrained peptides. Domain-sized compounds include high affinity binders based on: the Z domain of Protein A (Affibody), gamma-B-crystalline or ubiquitin (Affilin), lipocalin (Anticalins), C-type lectin (Atrimers), ankyrin repeats (DARPins), Fibronectin type 3 (FN3 scaffolds—Adnectins, Centyrins, Monobodies, Pronectins, Tn3), the SH3 domain (Fynomers), Serine protease inhibitor (Kunitz domains), the OB-fold domains (OBodies), the VHH domain (Nanobody), the scFV domain (CRAb), the PDZ domain (Affinity Clamp), or the sac7d DNA binding protein (Affitins). Constrained peptides include high affinity binders based on: the A-domain (the family of human extracellular receptor domains) (Avimers), bicyclic peptides, and Cys-knots. For each of these non-immunoglobulin high affinity scaffolds, binding to monoclonal antibody products is achieved through construction of a variant library followed by biopanning for those variants that bind the monoclonal antibody products, and finally one or more rounds of affinity maturation to achieve a high affinity binding. Because of the inherent variation in library construction, biopanning technique, and rounds of affinity maturity, final protein sequences can vary each time the process is undertaken by someone skilled in the art. Therefore, all such variations of the above non-immunoglobulin high affinity binders are included in the present disclosure. As an example, a variant library of the DNA binding protein Sac7d is created and then biopanned against a monoclonal antibody. The resultant sac7d variants are affinity matured using ribosome display technology. With successive rounds of affinity maturation, binding affinities are improved eventually creating a sac7d variant that binds a monoclonal antibody with a dissociation constant (K_{D}) of less than 10₋₇ M (SEQ ID NO: 5), for additional details WO2011077395A1 (Pecorari, 2011).

High affinity binders that bind monoclonal antibody products and are based on immunoglobulins or immunoglobulin fragments are also envisioned. Modifications to such an antibody-binding immunoglobulin or antibody-binding immunoglobulin fragment are envisioned. Such modifications include methods of enhancing binding affinity through structure based designed and/or affinity maturation techniques.

Non-peptide based high affinity binders that bind antibodies are also envisioned. Such synthetic, low molecular weight compounds include, but are not limited to, histidyl-Sepharose matrix, the synthetic affinity support based on dichloropyridine (Avid AL), divinylsulfone activated agarose (thiophilic gel) alone or bound to hetero-aromatic ligands, biomimetic dyes, a triazine scaffold with two spatially oriented substituents that mimic the helical twist of the key dipeptide in Protein A (such as, but not limited to, artificial Protein A, optimized IgG binding 22/8, or biomimetic protein L 8/7), or commercially available products such as MabSorbent A1P, MabSorbent A2P (Prometic Biosciences Ltd.), MEP HyperCel (Pall Life Sciences), Kaptiv-GY (Tecnogen), or FASTMabsA (UpFront Chromatography).

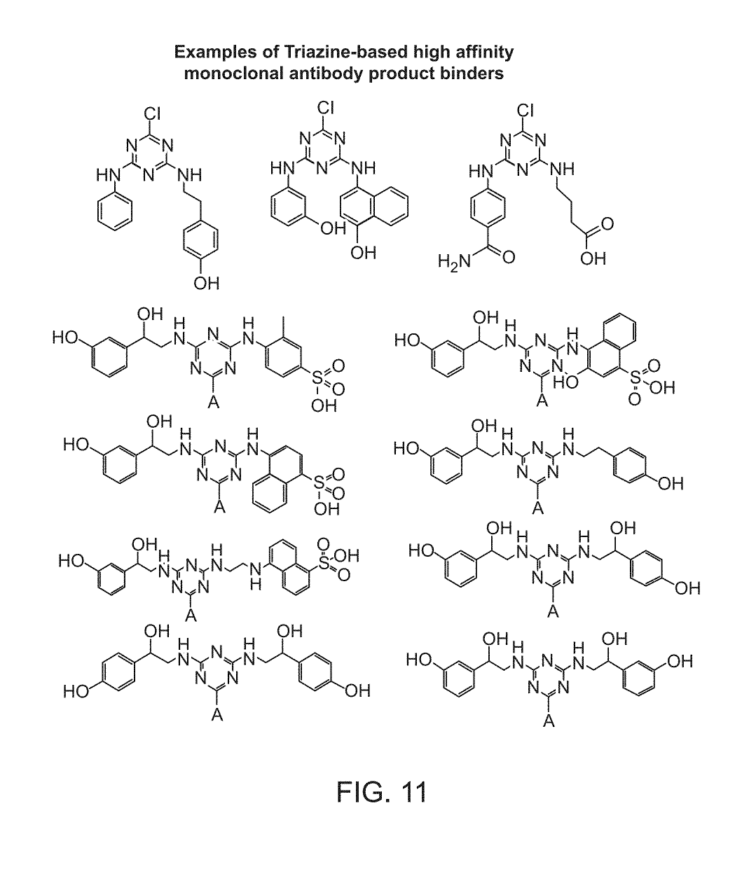

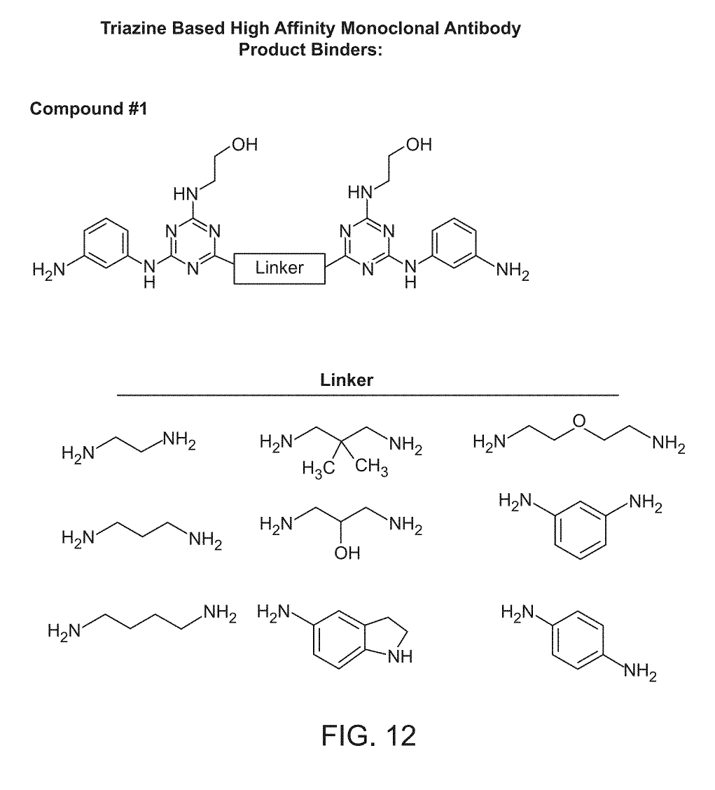

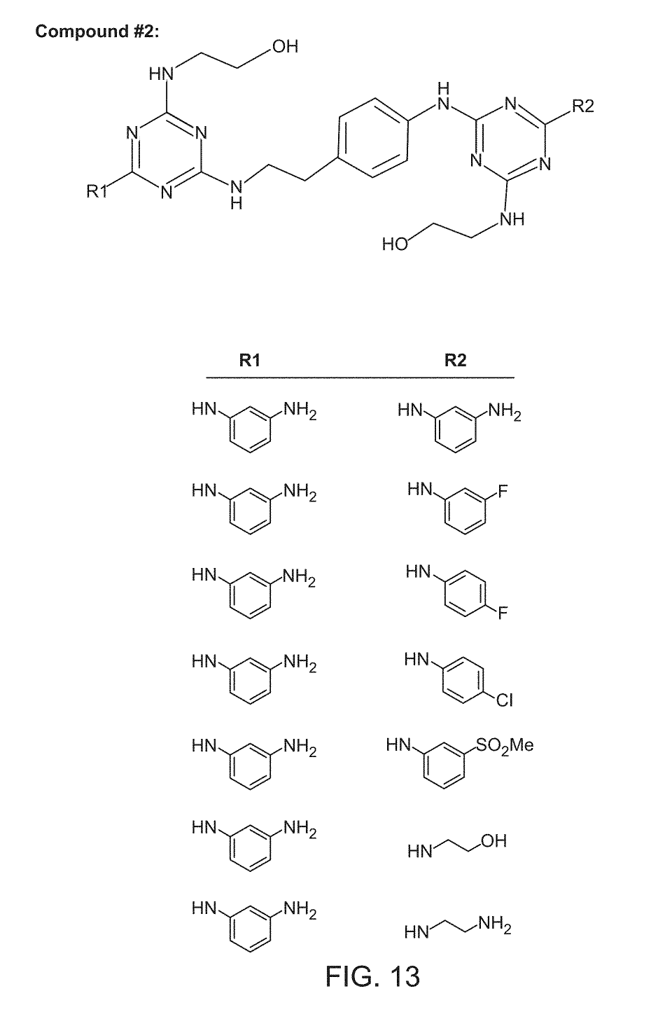

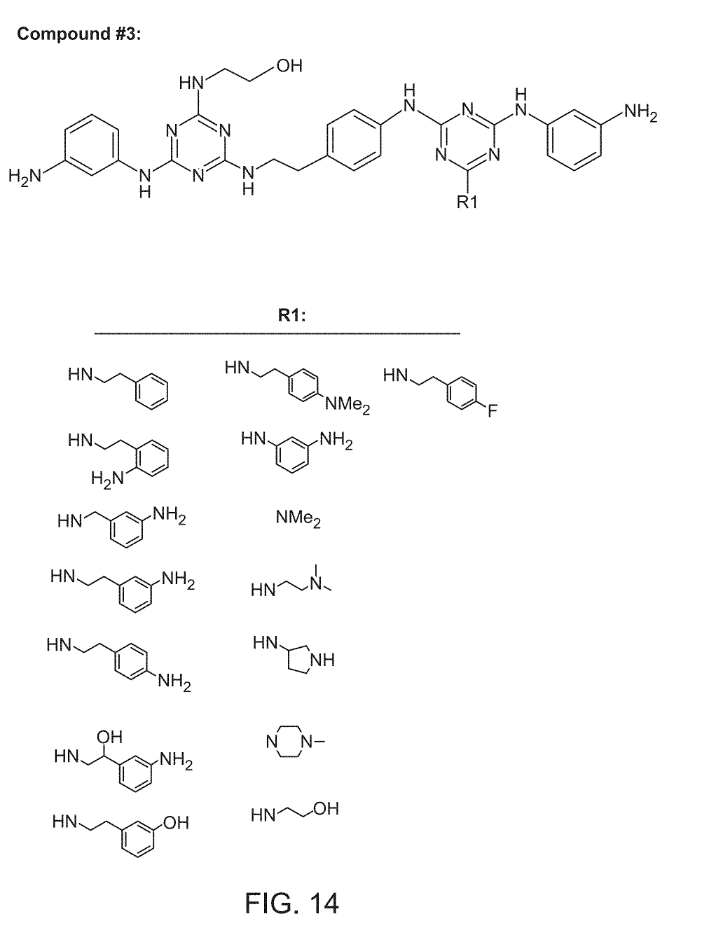

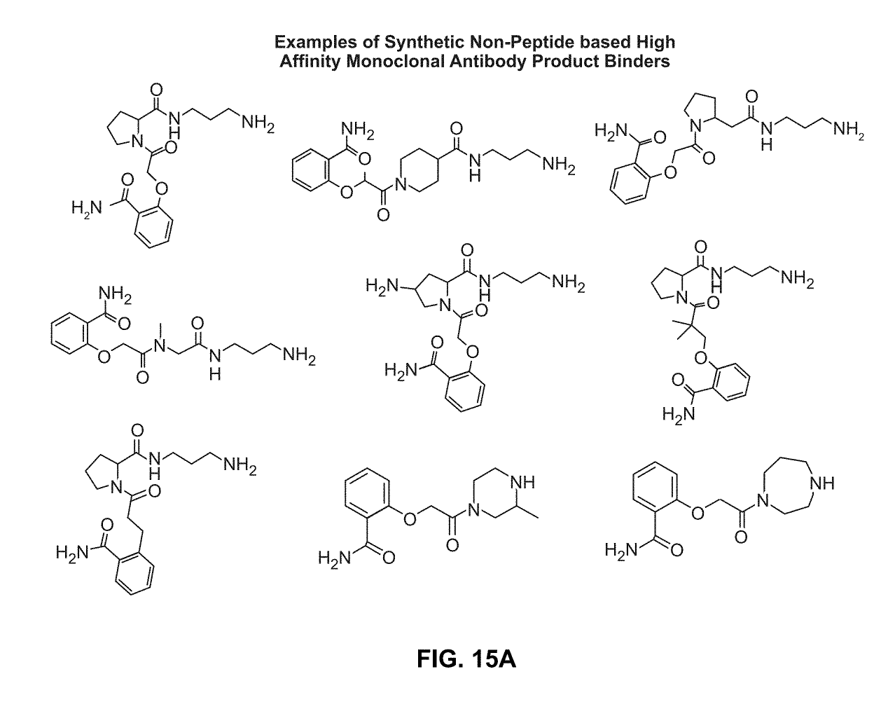

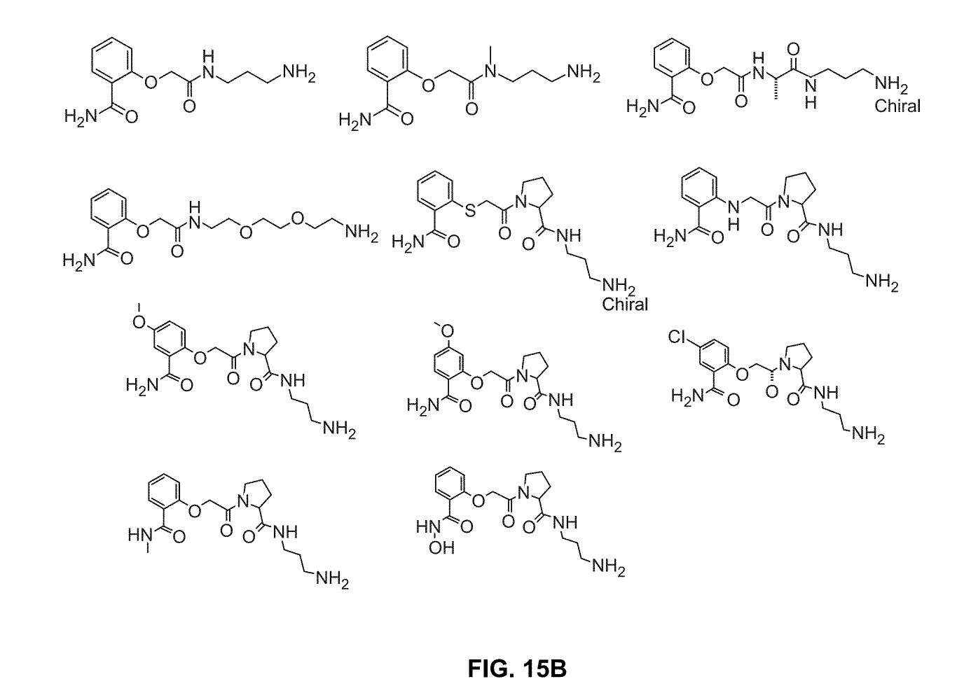

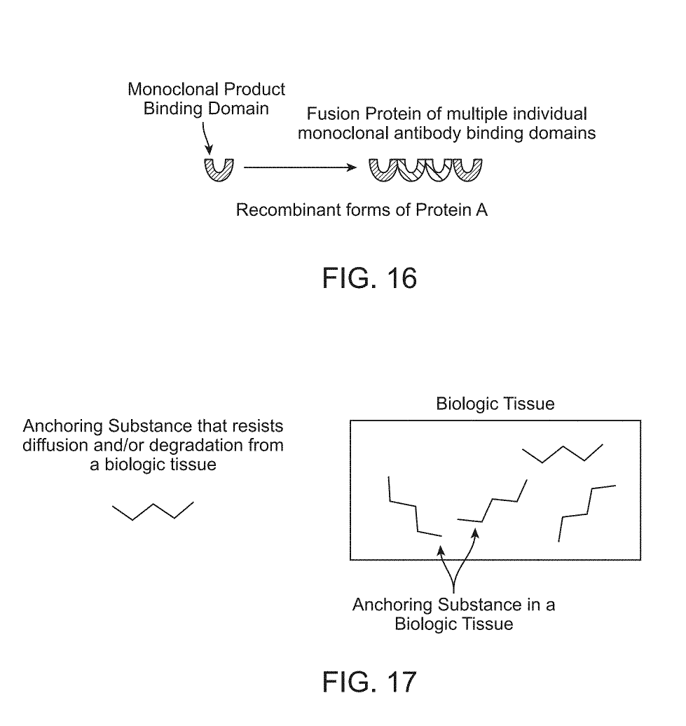

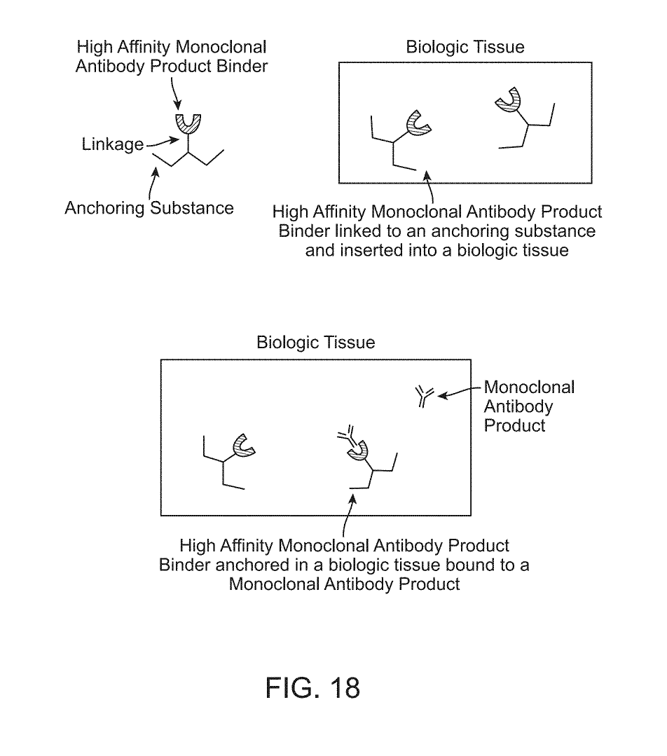

Non-peptide based binders of particular interest are triazine-based high affinity binders, for example as shown in FIGS. 11-15, see for example Zacharie, Boulos, et al. “2, 4, 6-trisubstituted triazines as protein a mimetics for the treatment of autoimmune diseases.” Journal of medicinal chemistry 53.3 (2010): 1138-1145, herein specifically incorporated by reference.

For any of the aforementioned compounds, a wide range of modifications can be envisioned. Such modifications include any changes in the primary, secondary, tertiary, or quaternary structure, or to the gene that expresses the protein or the post translational modifications of the protein or to the chemical structure of the compound. Such modifications also include the method of synthesis including chemical synthesis, or expression in prokaryotic or eukaryotic systems. Such modifications can be made to achieve or enhance a desired biologic affect, facilitate anchoring to a biologic tissue, resist degradation, enhance efficacy, improve solubility, facilitates synthesis, expression, or purification. Examples of affinity maturation methods include, but are not limited to, structure based design and affinity maturation with or without the use of ribosomal, phage, bacterial, or eukaryotic display techniques.

In another embodiment changes are made to enhance binding to a specific class or subtype of monoclonal antibody products, for instance human IgG1. In another embodiment, changes are made to resist proteolytic or chemical degradation within the biologic tissue including, but not limited to, substitution at the nitrogen atom to create protease-resistant peptides (peptoids), incorporation of alpha-methyl functionalized amino acids directly into the main chain during standard protein synthesis, insertion of or substitution with non-proteinogenic (“unnatural”) animo acids, inclusion of or substitution with non-natural beta-amino acids, inclusion of or substitution with non-natural beta-amino acids specifically before or after enzymatic cleavage sites as predicted by protease prediction software (such as PROSPER), substitution of alpha amino acids in an alpha helix with beta amino acids in an ααβ, αααβ, ααβαααβ, or αₙβₘ configuration, or unordered substitution of alpha amino acids with beta amino acids that maintains the secondary or tertiary structure and/or binding affinity, inclusion of or substitution with non-natural d-amino acids, inclusion of or substitution with d-amino acids at the terminal ends of a peptide sequence, full or partial sequence reversal with d-amino acids substitutions (known as retro-inverso peptides) or other non-natural amino acid substitutions in order to maintain the secondary or teritiary structure and/or binding, addition of hyaluronic acid, polyethylene glycol, or other hydrogel moieties. In another embodiment, changes are made to reduce an immunological response. In another embodiment, changes are made to inhibit cellular uptake. In another embodiment, changes are made to increase or reduce solubility. In another embodiment, changes are made to prevent diffusion out of or into a specific biologic tissue, for instance the retina or vitreous. In another embodiment, changes are made to prevent toxicity to a biologic tissue. In another embodiment, changes are made to prevent toxicity to intraocular structures. In another embodiment, inclusion of, single amino acids, groups of amino acids, linker sequences, or non-proteinogenic amino acids are used to facilitate binding to an anchoring substance or to facilitate binding directly to biologic tissue. Combinations of one or more of the aforementioned modifications are envisioned.

Any of the aforementioned compounds with or without any of the above modifications can be linked together to form a fusion compound, which includes fusion of one or more similar or dissimilar compounds.

An example of how these modifications can be used in conjunction is provided. Beginning with the native Protein A sequence, affinity maturation can be used to enhance the binding domain for monoclonal antibody product while eliminating the FAB binding and the insertion of non-proteinogenic amino acids to the C-terminus of the amino acid sequence can be added to facilitate anchoring to a biologic tissue. As another example, the native protein A sequence can be reduced to the Z-domain, a monoclonal antibody product binding domain that is composed of alpha helices and lacks disulfide bonds. This Z-domain can be further minimized though affinity maturation and structure based design. The subsequent minimized domain can be repeated forming a fusion protein of 2 or more minimized domains. This hybrid fusion protein can then be further modified to facilitate anchoring in a biologic tissue.

### Anchoring Region or Moiety

The aforementioned high affinity binders are joined to a region or moiety that provides for anchoring in/to a biologic tissue. The purpose of this anchor region or moiety is to retain the high affinity binder in the biologic tissue of interest and prevent its diffusion out of the tissue. Anchoring here broadly refers to any method of increasing the resident time of any of the aforementioned high affinity binders in a biologic tissue.

There are a number of methods for anchoring these high affinity binders in a biologic tissue, including, but not limited to: the high affinity binder can be bound to an anchor that restricts diffusion from the biologic tissue, the high affinity binder can be constructed such that its charge, size, polarity, or hydrophobicity limits its ability to diffuse from a biologic tissue (in effect the high affinity binder is itself the anchor), and/or the high affinity binder can be bound (anchored) to the biologic tissue through either covalent or non-covalent binding.

A substance that, when covalently or non-covalently bound to a high affinity binder increases its resident time in the biologic tissue of interest may be referred to as an anchoring region (for example of a polypeptide) or moiety (for example a chemical group such a guanidinium), or generically as a substance. An anchoring substance may be both locally degraded and diffuse out of a biologic tissue as long as either of these processes occur slower than does the degradation or diffusion of the high affinity binder.

TABLE 2

| SEQ | | |
|---|---|---|
| ID | | |
| 15 | GGGGS | Flexible |
| | | Linker |
| 16 | KESGSVSSEQLAQFRSLD | Flexible |
| | | Linker |
| 17 | EGKSSGSGSESKST | Flexible |
| | | Linker |
| 18 | GSAGSAAGSGEF | Flexible |
| | | Linker |
| 19 | EAAAK | Rigid |
| | | Linker |
| 20 | A(EAAAK)_{N} A | Rigid |
| | | Linker |
| 21 | (XP)_{N} | Rigid |
| | | Linker |
| 22 | GVYHREARSGKYKLTYAEAKAVCEFEGGHLATYK | LINK |
| | QLEAARKIGFHVCAAGWMAKGRVGYPIVKPGNCG | |
| | FGKTGIIDYGIRLNRSERWDAYCYNPHAK | |
| 23 | RYPISRPRKRC | HABP |
| 24 | RPSRPRIRYKC | HABP |
| 25 | LKQKIKHVVKLKVVVKLRSQLVKRKQN | HABP |
| 26 | STMMSRSHKTRSHHV (d-peptides) | HABP |
| 27 | GAHWQFNALTVRGGGS | HABP |

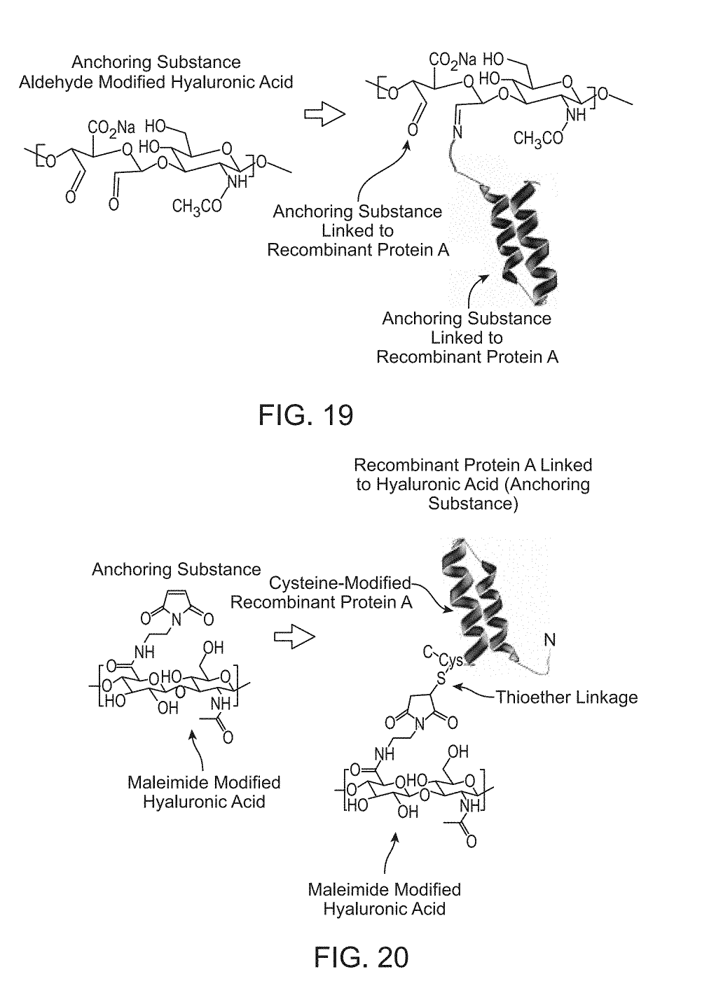

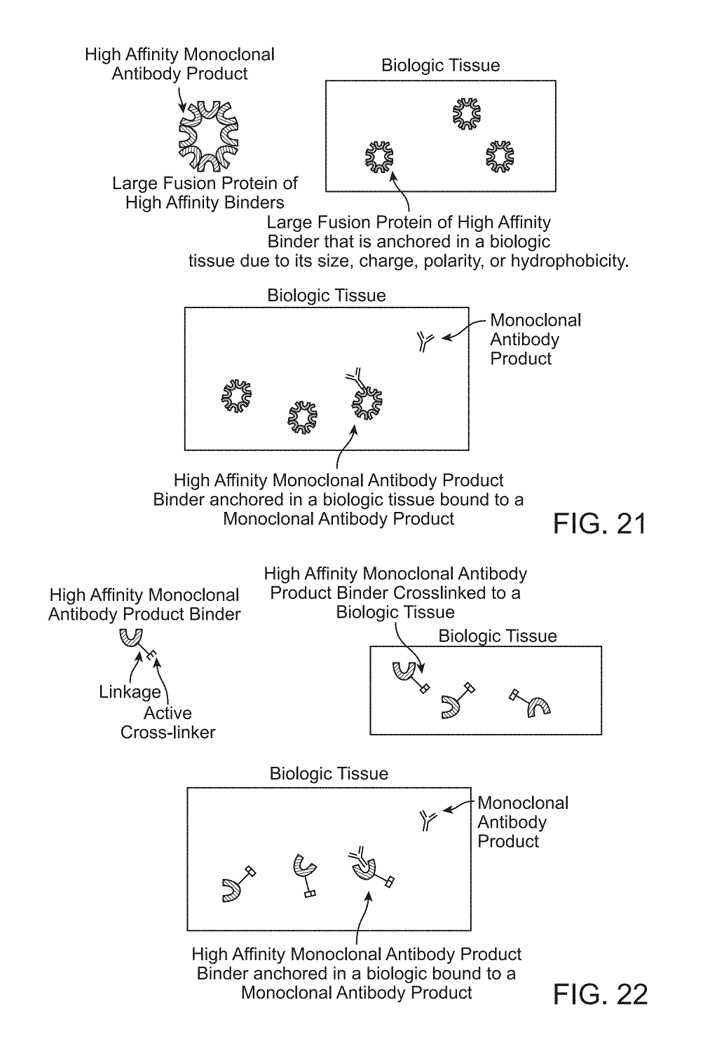

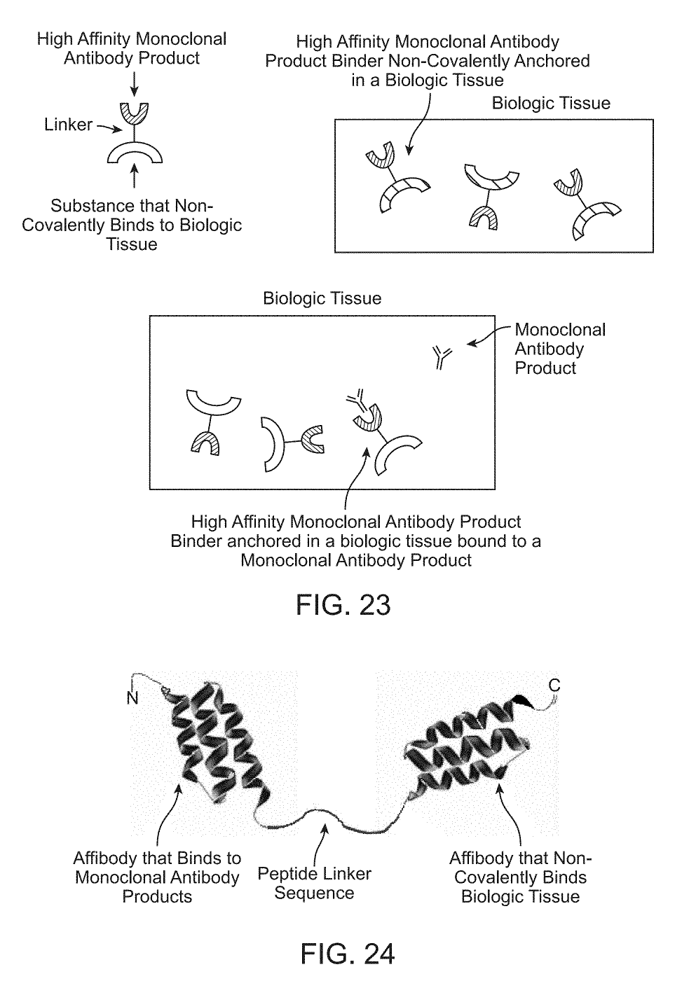

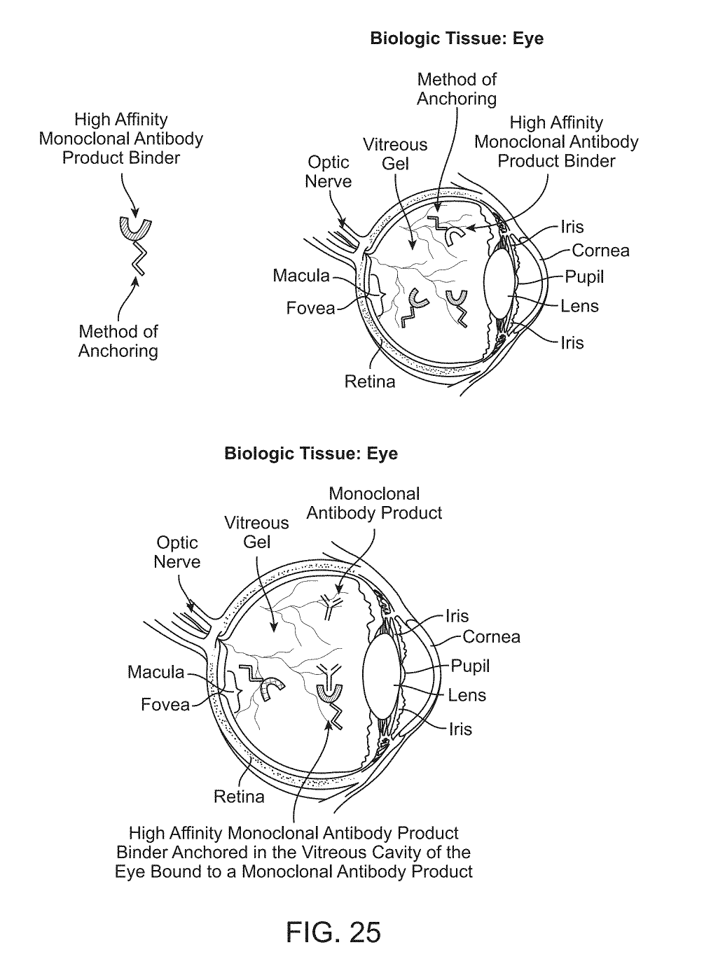

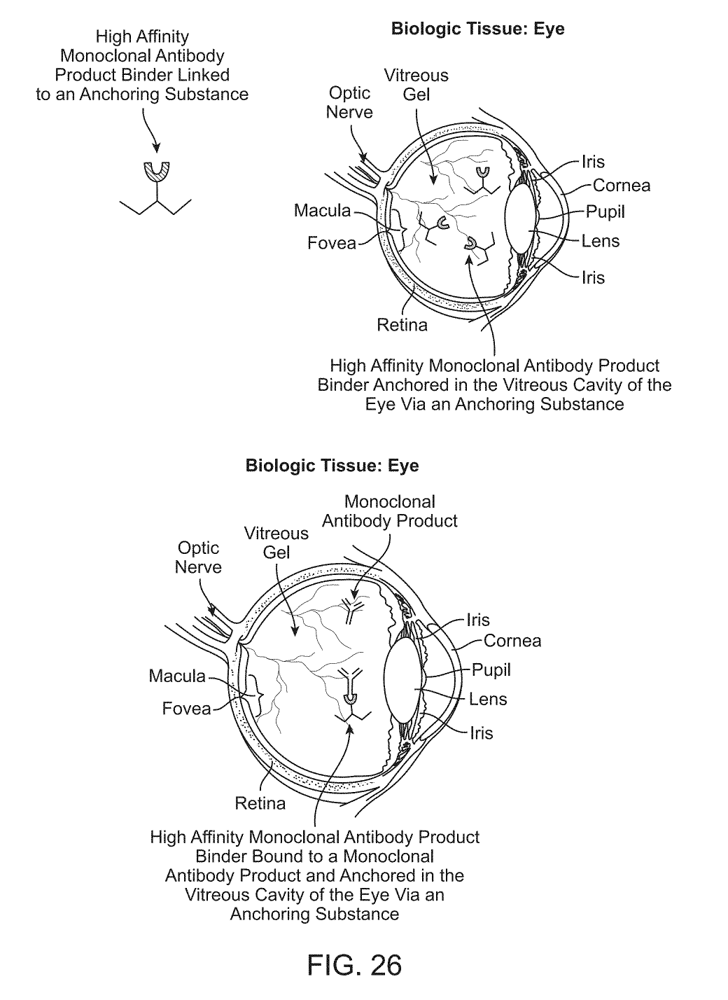

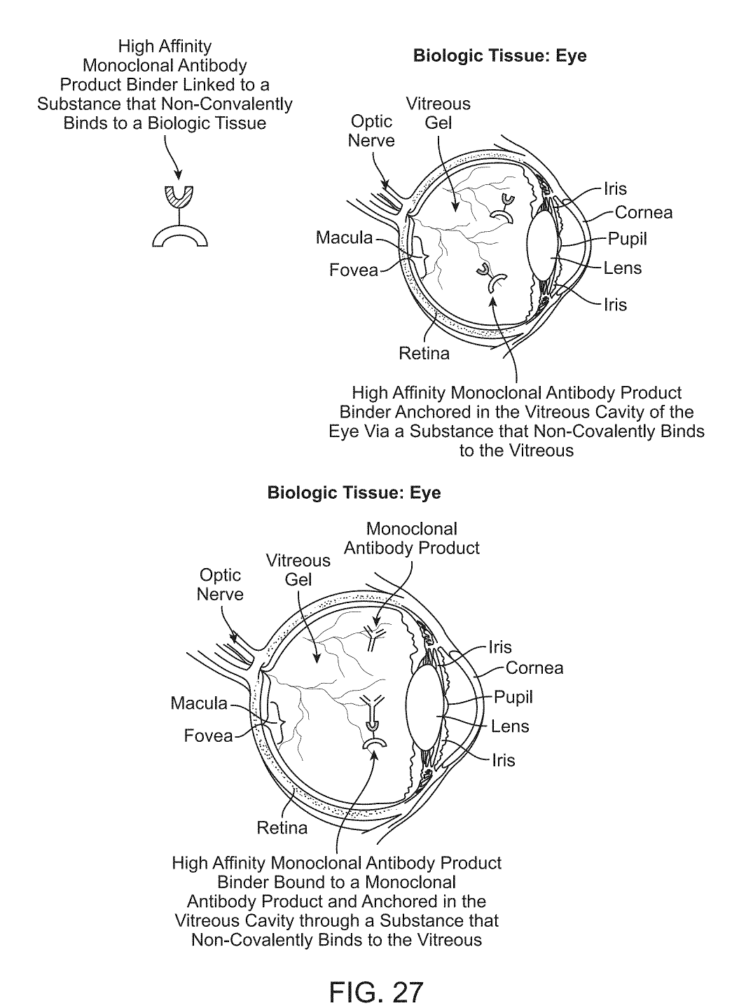

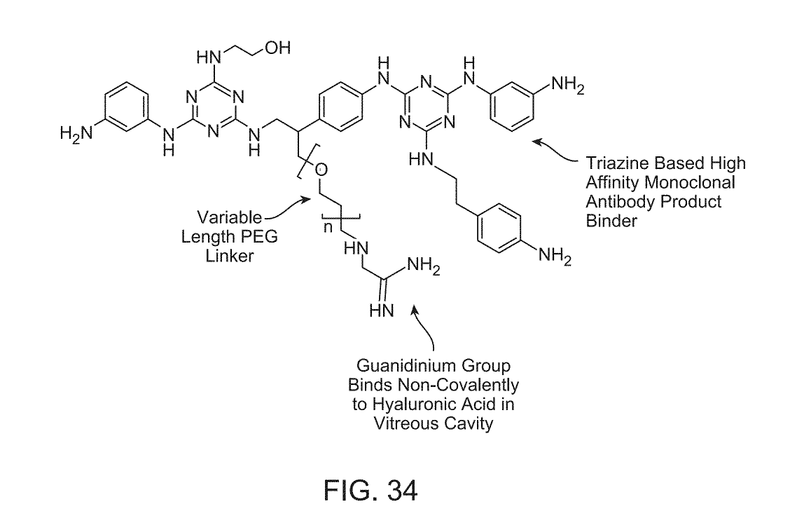

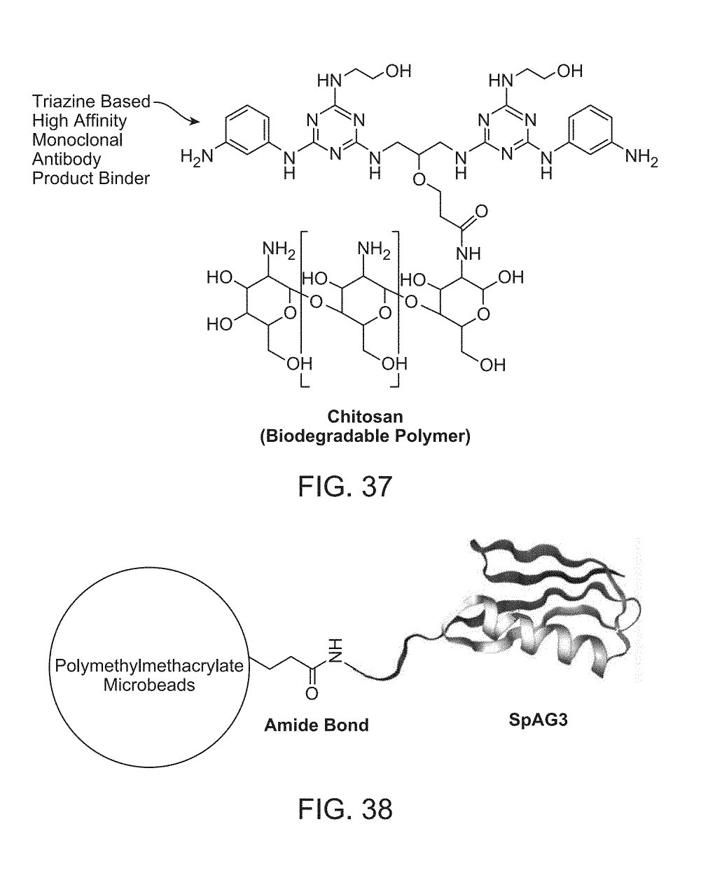

There are a number of different methods envisioned for anchoring a high affinity binder in the eye. The most exemplary among them are to anchor the high affinity binder to either the hyaluronic acid of the vitreous cavity or to anchor it to a polymer such as chitosan or crosslinked hyaluronic acid. An anchor may comprise a moiety that binds to hyaluronic acid at a high affinity, which moiety may comprise or consist essentially of, for example, a poly-arginine sequence, a poly-lysine sequence, a guanidinium group, a LINK domain (for example SEQ ID NO:22), an HA binding peptide (for example SEQ ID NO:23-27), etc. These can be linked through, for example, a variety of peptide linkers (examples SEQ ID NO: 15-21), or non-peptide linkers, such as a variable length polyethylene glycol (PEG) moiety, etc. The high affinity binder can be any of the previously mentioned high affinity binders with specific emphasis on the minimized Z-domain (SEQ ID NO: 1 and 2) and the triazine-based small molecules. An anchor that binds hyaluronic acid can be covalently linked to each of these high affinity binders (FIGS. 34-36). In other embodiments the anchor comprises an anchoring moiety or group that retains the anchor in the biological tissue of interest, e.g. where the anchor comprises a polymer that increases tissue retention. Polymers may include, for example, chitosan (FIG. 37), polymethacrylate microbeads (FIG. 38), an aldehyde modified hyaluronic acid (FIG. 19), maleimide-modified hyaluronic acid (FIG. 20), etc. See, for example, Wisniewski and Vilcek Cytokine & growth factor reviews 8.2 (1997): 143-156; and Amemiya et al. Biochimica et Biophysica Acta (BBA)-General Subjects 1724.1 (2005): 94-99, each herein specifically incorporated by reference.

For biologic tissue, including the eye, a variety of anchoring methods are also envisioned. In one embodiment, the anchoring substance used to anchor the high affinity binder is a natural polymer. Such natural polymers include, but are not limited to, glycosaminoglycans (e.g. heparan sulfate, chondroitin sulfate, keratan sulfate, hyaluronic acid), proteoglycans, collagen, alginate, cellulose, chitosan, xylan, and dextran. Combinations of these polymers or crosslinked polymers are envisioned. In another embodiment, the anchoring substance is a synthetic polymer. Examples of such synthetic polymers include, but are not limited to, poly(vinylpyrrolidone) (PVP), poly(2-hydroxyethylmathacrylate) (PHEMA), polyacylic acid (PAA), phosphoryl-based polymers, sulfobetaine/carboxybetaine polymers, poly(ethylene oxide) (PEO), poly(propylene oxide) (PPO), poly(ethylene glycol) (PEGylation), polyurethane, polystyrene, polysulfone, poly(lactic acid) (PLA), poly(glycolic acid) (PGA), poly(lactic-co-glycolic acid) (PLGA), poly(glycolide-co-caprolactone), and poly(glycolide-co-trimethylene carbonate). Combinations of or modifications to the above natural and synthetic polymers are also envisioned, for example, the triblock polymer PEO—PPO-PEO. In another embodiment, the anchoring substance is a microsphere or bead composed of either a natural or synthetic polymer, or a combination thereof.

In another embodiment, the anchoring substance is a hydrogel, organogel or nanocomposite hydrogel.

In another embodiment, the anchoring substance is a nanoparticle or nanotube. The composition of such a nanoparticle or nanotube includes, but is not limited to, gold, silica, carbon, iron oxide, quantum dot, dendrimer, liposomes, N-(2-hydroxypropyl) methacrylamide (HPMA), hydroxylapatite.

In another embodiment, the anchoring substance is a large protein or group of smaller proteins that either are resistant to degradation and/or have restricted diffusion due to their size, charge or polarity.

In another embodiment, the anchoring substance is a biomaterial that is injected or implanted into the biologic tissue of interest. Any biomaterial that does not illicit an inflammatory reaction and has a reduced or absent rate of diffusion from the biologic tissue of interest is considered. Such a biomaterial could be in the form of an implantable device. Examples of biomaterials that can serve as an anchoring substance include, but are not limited to, polymers, polyurethanes, silicones, fluorinated biomaterials, acrylics, titanium, nitinol (NiTi), stainless steel, ceramics, glasses, glass-ceramics, natural hydroyxapatites, synthetic hydroxyapatites, alumina, hydrogels, degradable/resorbable biomaterials, pyrolytic carbon, “smart polymers”, medical fibers, biotextiles, textured materials, porous materials, biomimetic materials, microparticles, and nanoparticles.

A number of methods of attachment between high affinity binder and an anchoring substance are envisioned. These can either be covalent or non-covalent attachments. Covalent attachments between an anchoring substance and high affinity binders can occur through a variety of chemical crosslinking reactions that utilize reactive amino acids functional groups, non-proteinogenic amino acids, or linkers at the N- or C-terminus of the high affinity binder. A wide range of chemical covalent crosslinking reactions can be used depending on the anchoring substance chosen. Examples of such covalent linkages include, but are not limited to, those containing polyethylene glycol (PEG), thioethers, disulfide bonds, amide bonds, carbon-nitrogen double bonds, and linkages generated by cycloaddition. Bifunctional, trifunctional, polyfunctional, and polyethylene glycol covalent crosslinkers can also be used. Peptide linkers include, for example, those disclosed by Chen et al. Advanced drug delivery reviews 65.10 (2013): 1357-1369, herein specifically incorporated by reference.

In another embodiment, covalent protein affinity tags are used. These include, but are not limited to Isopeptag, Spytag, Snooptag.

Non-covalent attachments between anchoring substance and a high affinity binder are also envisioned. For instance, the high affinity binder can have an affinity tag, which non-covalently binds to proteins, amino acid sequences, or metal matrices on the anchoring substance. Examples include, but are not limited to biotin-avidin, biotin-strepavidin, biotinneutravidin, avitag, calmodulin-tag, polyglutamate-tag, E-tag, Flag-tag, HA-tag, His-tag, Myc-tag, NE-tag, S-tag, SBP-tag, Softag 1, Softag 3, Strep-tag, TC tag, V5 tag, VSV-tag, Xpress tag, BCCP, Glutathione-S-transferase tag, Green Fluorescent protein-tag, Halo-tag, Maltose binding protein-tag, Nus-tag, Thioredoxin-tag, Fc-tag.

To limit diffusion, a high affinity binder can be constructed such that its physical characteristics limit its ability to diffuse from a biologic tissue of interest. In one embodiment, the charge of the high affinity binder in a physiologic pH is adjusted such that it restricts the high affinity binder from diffusing from the biologic tissue. In another embodiment, the size of the high affinity binder is increased such that it restricts diffusion. This can be achieved by creating a fusion protein of a number of repeating units of similar or dissimilar high affinity binders. For instance, the minimized Z domain, a recombinant form of Protein A, can be covalently linked to itself form a fusion protein of any number of repeating Z-domains. The resultant fusion protein is larger and has restricted diffusion out of a biologic tissue. In another embodiment, the high affinity binder has an altered polarity such that its diffusion from a biologic tissue is limited. In another embodiment, the high affinity binder is hydrophobic such that it is less soluble in water and therefore diffuses more slowly from a biologic tissue.

Anchoring a high affinity binder by binding it to a biologic tissue. Another approach to anchoring a high affinity binder in a biologic tissue is to bind the high affinity binder to the biologic tissue of interest through either covalent or non-covalent binding.

Active covalent bifunctional or multifunctional crosslinkers can be used to covalently directly attach a high affinity binder to a biologic tissue, thereby anchoring it in the tissue. Active crosslinkers which target carboxyl-to-amine reactive groups, amine-reactive groups, sulfhydryl-reactive groups, aldehyde-reactive groups, photoreactive groups, or hydroxyl-reactive groups can be used to bind and anchor recombinant protein A directly to a biologic tissue. Examples of specific crosslinkers include, but are not limited to carbodiimide, NHS ester, imidoester, pentafluorophenyl ester, hydroxymethyl phosphine, maleimide, haloacetyl pyridyldisulfide, thiosulfonate, vinylsulfone, hydrazide, alkoxyamine, diazirine, aryl azide, Isocyanate. To prevent autocrosslinking, that is crosslinking between molecules of the high affinity binder, the high affinity binder can be modified such that lacks functional groups specific for the crosslinking agent. For instance, the Z-domain lacks sulfhydryl-reactive groups. Therefore, a malemide crosslinker can be covalently attached to the Z-domain and is unable to crosslink other Z-domains. Only when the Z-domain-maleimide crosslinker fusion is inserted into a biologic tissue containing sulfhydryl groups will the crosslinker be active. The result is crosslinking of the Z-domain through the maleimide crosslinker directly to the biologic tissue of interest.

Non-covalent methods of anchoring a high affinity binder rely on an intermediary which is both linked to the high affinity binder and is also capable of noncovalently binding a biologic tissue of interest. Such intermediaries can be either protein-based or non-protein based. Examples of non-protein based anchoring methods are of strongly positive (basic) side chains such as guanidinium, which associates through ionic interactions with strongly negative glycosaminoglycans such as hyaluronic acid in the vitreous cavity. Examples of protein-based anchoring methods include, but are not limited to, those using immunoglobulins, immunoglobulin fragments, non-immunoglobulin affinity proteins, or short high affinity peptide sequences. Any immunoglobulin or fragment thereof, directed against any component of a biologic tissue is envisioned as an example of a protein-based anchoring method. For instance, using the vitreous cavity of the eye as an example, an immunoglobulin that binds hyaluronic acid or type 2 collagen can be envisioned as anchoring moiety that can be attached to a high affinity binder for use in the vitreous cavity. In this instance, one of the aforementioned high affinity binders is linked to an immunoglobulin against hyaluronic acid. When this compound (the high affinity binder linked to the immunoglobulin) is inserted into the vitreous cavity, the result is anchoring of the high affinity binder through the anchoring moiety to endogenous hyaluronic acid.

Similarly, any non-immunogloblin affinity protein that has been designed to bind to a biologic tissue of interest is envisioned. Examples of such non-immunoglobulin affinity proteins include, but is not limited to, domain-sized compounds and constrained peptides. Domain-sized compounds include high affinity binders based on: the Z domain of Protein A (Affibody), gamma-B-crystalline or ubiquitin (Affilin), lipocalin (Anticalins), C-type lectin (Atrimers), ankyrin repeats (DARPins), Fibronectin type 3 (FN3 scaffolds—Adnectins, Centyrins, Monobodies, Pronectins, Tn3), the SH3 domain (Fynomers), Serine protease inhibitor (Kunitz domains), the OB-fold domains (OBodies), the VHH domain (Nanobody), the scFV domain (CRAb), or the PDZ domain (Affinity Clamp). Constrained peptides include high affinity binders based on: the A-domain (the family of human extracellular receptor domains) (Avimers), bicyclic peptides, and Cys-knots. For each of these non-immunoglobulin high affinity protein classes, binding to a biologic tissue of interest is achieved through construction of a variant library followed by biopanning for those variants that bind to components within the biologic tissue of interest with one or more rounds of affinity maturation to achieve binding affinities sufficient to anchor the high affinity binder within the biologic tissue.

Such methods also include forming recombinant forms of DNA that when expressed contain both the protein based anchoring compound and the high affinity monoclonal antibody product binder with or without an intervening linking sequence. Such methods also include chemically synthesizing both the high affinity monoclonal antibody product binder with either a protein-based or non-protein based anchoring method.

Dual non-covalent and covalent methods of anchoring are also envisioned. For instance, in one embodiment, an unnatural electrophile is placed adjacent to the non-covalent target binding site on the non-covalent anchoring compound. The result is that when the noncovalent anchoring substance binds to a biologic tissue, the unnatural electrophile interacts with cysteine, lysine, or histidine in the biologic tissue and forms a covalent bond. This results in dual non-covalent and covalent anchoring to the biologic tissue. As an example, an affibody against type II collagen in the vitreous is generated. A weak electrophile, acrylamide, is placed near the type II collagen binding site on the affibody. This affibody is linked to a high affinity monoclonal antibody product binder. When this compound is inserted into the vitreous cavity, the affibody non-covalently binds to type II collagen, acrylamide then creates a covalent attachment to type II collagen. The result is dual non-covalent and covalent anchoring. Any weak electrophile can be envisioned and can be attached to any non-covalent method of attachment. Such dual non-covalent and covalent anchoring techniques can be used to anchor to any component of a biologic tissue including, but not limited to, proteins, glycosaminoglycans, or lipids.

Labelling of either the high affinity monoclonal antibody product binder or the anchoring substance. The high affinity monoclonal antibody product binder or the anchoring substance can be labelled such that its rate of diffusion or degradation from a biologic tissue can be monitored. Labelling can be through either covalent or non-covalent binding. Labelling can use fluorescent tags such as organic dyes, biologic fluorophores, or quantum dots. Examples of organic dyes include, but are not limited to FITC, TRITC, DyLlght Fluors. Examples of biologic fluorophores include, but are not limited to green fluorescent protein (GFP), RPhycoerythrin. Labelling can use enzyme conjugates that rely on either a colorimetric, chemiluminescent, or fluorescent signal output. In another embodiment, metabolic labelling can be used to label the high affinity binder by synthesizing it or expressing it with fluorescently or radiolabeled amino acids. In another embodiment, biotin is added to the high affinity binder or the anchoring substance and non-covalently binds to avidin, streptavidin, or neutravidin that has either a fluorescent or radiolabel.

### Methods of Use

Effective doses of the combined agents of the present invention for therapeutic effect vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage of each agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. In some embodiments, the therapeutic dosage of each agent is not based on body weight. In some embodiments, the dosage of the anchor is dependent on the dosage of the therapeutic monoclonal antibody. An exemplary treatment regime entails administration once every two weeks or once a month or once every 3 to 6 months, or once every 6 to 12 months. Therapeutic entities of the present invention are usually administered on multiple occasions. Intervals between single dosages can be weekly, monthly or yearly. Intervals can also be irregular as indicated by measuring levels of the therapeutic entity in the patient. Alternatively, therapeutic entities of the present invention can be administered as a sustained release formulation, in which case less frequent administration is required. Dosage and frequency vary depending on the half-life of the anchor and therapeutic antibody in the patient.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Compositions can be administered by intravitreal, intra-articular, intra-tumoral, subcutaneous, intracranial, intraperitoneal, intramuscular, etc. means, typically by direct injection into the targeted tissue.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The pharmaceutical compositions are generally formulated as sterile, substantially isotonic and in full compliance with all Good Manufacturing Practice (GMP) regulations of the U.S. Food and Drug Administration.

Toxicity of the combined agents described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The pharmaceutical compositions can be administered in a variety of unit dosage forms depending upon the method of administration. The compositions for administration will commonly comprise an antibody or anchor dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the anchor agents and formulations thereof, of the invention and instructions for use. The kit can further contain a least one additional reagent, e.g. a therapeutic antibody, etc. Kits typically include a label indicating the intended use of the contents of the kit. The term label includes any writing, or recorded material supplied on or with the kit, or which otherwise accompanies the kit.

The compositions can be administered for therapeutic treatment. Compositions are administered to a patient in an amount sufficient to achieve the desired result, as described above. An amount adequate to accomplish this is defined as a “therapeutically effective dose.”, which may provide for an improvement in overall survival rates, improvement of vision, etc. Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

### EXAMPLES

### Example 1

Recombinant Protein A is crosslinked to aldehyde modified hyaluronic acid using a reductive amination. Recombinant protein A is dissolved in a solution of 0.1M sodium citrate, 0.05M sodium carbonate at pH 10. The dissolved recombinant protein A is added to aldehyde modified hyaluronic acid. The reaction occurs over four to six hours to maximize protein immobilization. The pH is adjusted to 7.2 and cyanoborohydride solution (5M NaCNBH₃ in 1M NaOH) is added to the reaction to reduce the Schiff Base and form a stable covalent amide bond. The solution is then washed with 1M Tris buffer to convert any unused aldehyde functional groups into hydroxyl groups. The result is recombinant protein A covalently linked to hyaluronic acid which can then be processed and sterilized for injection into the eye (or another biologic tissue) for the purposes of increasing the resident time of full or modified antibodies, Fab fragments or proteins with Fc regions.

Alternatively the Z-domain of protein A is conjugated to an anchoring moiety. To facilitate its use, the Z-domain of protein A can be further modified with a cysteine domain at its C- or N-terminus. Hyaluronic acid is then modified with iodoacetamide, maleimide, chloroacetamide, or thiol. The Z-domain of protein A is crosslinked to hyaluronic acid through, for example, covalent cross-linking of the cysteine residue and the maleimide group via a thiol Michael's addition reaction.

### Example 2

The Z-domain of protein A has a dissociation constant (K_{D}) less than 10⁻⁸ M to monoclonal antibody products. The Z-domain of Protein A is further minimized through structure based design and/or affinity maturation to arrive at a truncated protein sequence, for instance: (SEQ ID NO:1) FNMQQQRRFYEALHDPNLNEEQRNAKIKSIRDD. To anchor this minimized version of the Z-domain of Protein A to a biologic tissue, it is conjugated to an anchoring moiety.

An example of an anchoring moiety is an affibody that non-covalently binds the biologic tissue. To achieve this, an affibody library of millions or more random variants is created. The affibody library is then screened against a target molecule, in this example, the vitreous cavity of the eye. A molecule from the vitreous cavity is then used as the target molecule for the affibody screen. For this example, hyaluronic acid is chosen.

A biopanning process is used to identify those affibody variants that bind to hyaluronic acid. Both the peptide and nucleic acid sequences of the affibody variants that bind hyaluronic acid are identified. Using a method of affinity maturation, for example ribosome display, one or more of these sequences is serially modified to further enhance binding to hyaluronic acid. Once binding is sufficiently high to facilitate anchoring in the biologic tissue, the final nucleic acid sequence is identified. The nucleic acid sequence from the minimized version of the Z-domain of Protein A is then linked to the nucleic acid sequence from the affibody to hyaluronic acid with or without an intervening nucleic acid sequence encoding a peptide linker sequence, such as a gly/ser linker (SEQ ID NO:17) (GGGS)_{N}. The resultant nucleic acid sequence is then used to generate a protein through either a protein expression system or through chemical synthesis. The resultant compound contains the minimized form of the Z-domain of Protein A and the affibody against hyaluronic acid, optionally separated by a linker sequence.

This compound is inserted into the vitreous cavity of the eye where the portion of the compound containing the minimized form of the Z-domain of Protein A binds monoclonal antibody products while the portion of the compound containing the affibody to hyaluronic acid results in non-covalent binding to endogenous hyaluronic acid.

The result is a compound that 1) binds monoclonal antibody products with a dissociation constant (K_{D}) less than 10₋₆ M (K_{D}<10₋₆ M), and 2) is anchored in a biologic tissue (in this example, the vitreous cavity of the eye).

### Example 3

The anchor comprises an ankyrin repeat protein (DARPin) is a method of anchoring the minimized version of the Z-domain of Protein A in a biologic tissue. In this example, the biologic tissue is the skin dermis.

A DARPin library of millions or more random variants is created. The DARPin library is then screened against a target molecule. In this example, the dermis of the skin is selected as an example of a biologic tissue. A molecule from the skin dermis is then used as the target molecule for the DARPin screen. For this example, type I collagen is chosen.

A biopanning process is used to identify those DARPin variants that bind to type I collagen. Both the peptide and nucleic acid sequences of the DARPin variants that bind type I collagen are identified. Using a method of affinity maturation, for example phage display, one or more of these sequences is serially modified to further enhance binding to type I collagen. Once binding is sufficiently high to facilitate anchoring in the biologic tissue, the final nucleic acid sequence is identified. The nucleic acid sequence from the minimized version of the Z-domain of Protein A is then linked to the nucleic acid sequence from the DARPin to type I collagen, with or without an intervening nucleic acid sequence encoding a peptide linker sequence, such as a gly/ser linker (SEQ ID NO:17) (GGGS)_{N}. The resultant nucleic acid sequence is then used to generate a protein through either a protein expression system or through chemical synthesis.

The resultant compound contains the minimized form of the Z-domain of Protein A and the DARPin against type I collagen, possibly separated by a linker sequence. This compound is then inserted into the dermis of the skin where the portion of the compound containing the minimized form of the Z-domain of Protein A binds monoclonal antibody products while the portion of the compound containing the DARPin to type I collagen results in non-covalent binding to endogenous type I collagen.

The result is a compound that 1) binds monoclonal antibody products with a dissociation constant (K_{D}) less than 10⁻⁶ M (K_{D}<10⁻⁶ M), and 2) is anchored in a biologic tissue (in this example, the dermis of the skin).

### Example 4

A triazine scaffold with two spatially oriented substituents that mimic the helical twist of the key dipeptide in Protein A can be used. Examples of compounds are shown in FIGS. 11, 12, 13, 14 and 15. Specifically, Compound 1 binds to monoclonal antibody products with a dissociation constant (K_{D}) less than 10⁻⁶ M (K_{D}<10⁻⁶ M). Structure based design is used to make modifications to this triazine scaffold to further reduce its dissociation constant with monoclonal antibody products. The triazine scaffold is then covalently linked to an active NHS ester. When the triazine scaffold is inserted into a biologic tissue, the active NHS ester crosslinks to local proteins, covalently linking the triazine scaffold in the biologic tissue. The result is the triazine scaffold binds monoclonal antibody products and because of anchoring to the biologic tissue through the NHS ester, the triazine scafford retains the antibody in the biologic tissue.

The anchoring moiety can be attached through a linker, for example see US 20020182172 A1, with a structure as shown below:

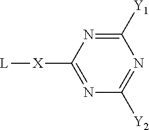

wherein:

L is the point of attachment to the polymer backbone;

X is a linker; and

Y₁ and Y₂ are each independently amino, substituted amino, C1-6alkyl, substituted C1-6alkyl, aryl, or substituted aryl.

### Example 5

In situ modeling of the effect of an anchored high affinity monoclonal antibody product binder on the duration of Bevacizumab (Avastin) injected into the vitreous cavity of the eye was performed, as shown in FIG. 28.

A two-compartment model of the eye was created using Matlab R2016a (The Mathworks, Inc, Natick, Massachusettes, United States). The Model has three species in the vitreous cavity of the eye: A (Avastin), Binder (Anchor), and IgG (Endogenous IgG present in the vitreous cavity. Model assumptions: Vitreous Volume: 4 mL, Initial Bevacizumab Concentration: 3.3E-7(M), IgG Concentration: 3.3E-8(M), Elimination Anchor (ke): 0.0023, Elimination Bevacizumab (ke): 0.09. The model was run with five different starting conditions to determine the required physical characteristics of an effective anchor: (A) No anchor present, (B) Initial Anchor Concentration: 3.3E-8(M), Kd˜1.0E-7, (C) Initial Anchor Concentration: 3.3E-7(M), Kd˜1.0E-8, (D) Initial Anchor Concentration: 3.3E-7(M), Kd˜1.0E-6, (E) Initial Anchor Concentration: 3.3E-7(M), Kd˜1.0E-6. When there is no anchor, bevacizumab is rapidly cleared from the vitreous cavity, falling below the minimum inhibitory concentration at approximately 45 days (A). When initial anchor concentrations similar to initial bevacizumab concentrations, an Kd˜1.0E-6 does not appreciably increase the retention above 45 days (data not shown), while a Kd˜1.0E-7 results in a retention to 80 days (C), and a Kd˜1.0E-8 results in an retention to over 180 days. If the initial anchor concentration is increased from 3.3E-7 to 3.3E-6 (10 times higher than the bevacizumab concentration), then a Kd˜1.0E-6 results in retention to over 100 days (E) and a Kd˜1.0E-7 results in retention to over 180 days (F). Based on these results an intravitreally injected anchor needs a dissociation constant of around Kd˜1.0E-6 or smaller, and ideally 1.0E-7 or 1.0E-8 or smaller.

Based on these results, candidate high affinity monoclonal antibody binders were selected with high binding affinities and dissociation constants (K_{D}) less than 10₋₆ M (SEQ ID NO: 1-14).

In vitro survival of ARPE-19 cells exposed to protein A was determined, as shown in FIG. 29. ARPE-19 cells were grown in Dulbecco's Modified Eagles medium (DMEM)/F12 with 10% FBS and plated at 4600 cells/well in 96 well plates on day 0. To each well was added 0.1 mL of growth media or protein A dissolved in growth media. Control cells doubled several times before reaching confluence at viability test on day 3. Cell viability was measured in triplicate using Alamar blue dye (560 nm/590 nm). Percentage survival is a comparison of fluorescent vales of treatment (growth media with protein A) versus control (growth media only). Protein A showed low cytotoxicity at concentrations of 0.1 mg/mL or lower. These safe concentrations are significantly greater than what is needed for anchoring monoclonal antibody products in the vitreous cavity.

Due to the low cytotoxicity of Protein A on ARPE-19 cells, Protein A, the Z-domain (SEQ ID NO: 3), and the minimized Z-domain (SEQ ID NO:1) were tested for in vivo biocompatibility. In Vivo Biocompatibility Histopathologic Results are shown in FIG. 30. Fourteen New Zealand White (NZW) Rabbits were used to assess in vivo biocompatibility of Protein A, the Z-domain (SEQ ID NO:3), and a minimized form of the Z-domain (SEQ ID NO: 1). On Day 1, each rabbit received an intravitreal injection of 0.05 mL of phosphate buffered saline in the right eye of 0.05 mL of the test article in the left eye. On Day 8, each rabbit received a repeat intravitreal injection of 0.05 mL of phosphate buffered saline in the right eye and 0.05 mL of the test article in the left eye. Slit lamp and dilated funduscopic exams were performed at days 1, 8, and 14. On day 14, eyes were enucleated, fixed in 10% formalin, sectioned and stained with H&E for histopathologic examination. (A) Control eyes showed no evidence of inflammatory infiltration in the cornea (top), anterior chamber (not shown), angle (middle), or vitreous/retina (bottom). Both (B) Protein A (0.31 mg/eye) and (C) the Z-domain (0.35 mg/eye) (SEQ ID NO: 3) (C) showed significant inflammatory infiltrate in the cornea (top), anterior chamber (not shown), angle (middle) and vitreous retina (bottom). Both a (D) high (0.28 mg/eye) and (E) low (0.14 mg/eye) intravitreal dose of the minimized version of the Z-domain (SEQ ID NO: 1) showed no inflammatory infiltration in either the cornea (top), anterior chamber (not shown), angle (middle), or vitreous/retina (bottom). External examination was consistent with this lack of intraocular inflammation and showed no difference between the control and minimized Z-domain treated rabbits. The minimized Z-domain showed good in vivo biocompatibility.

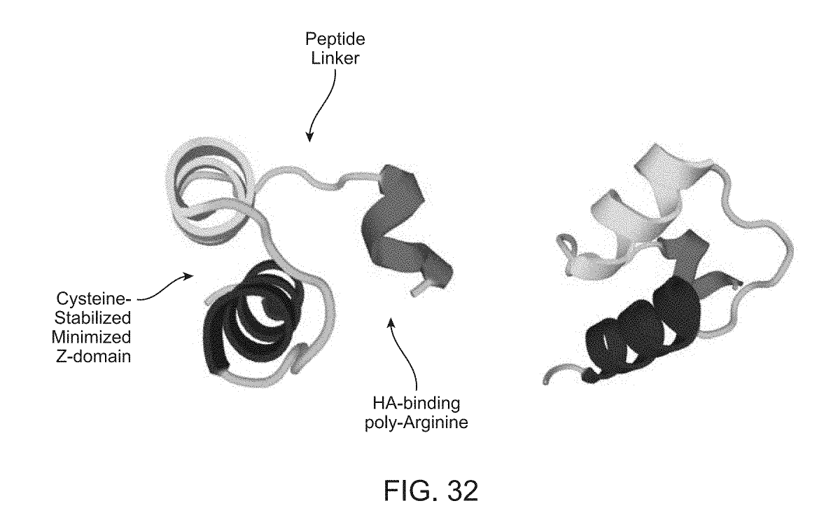

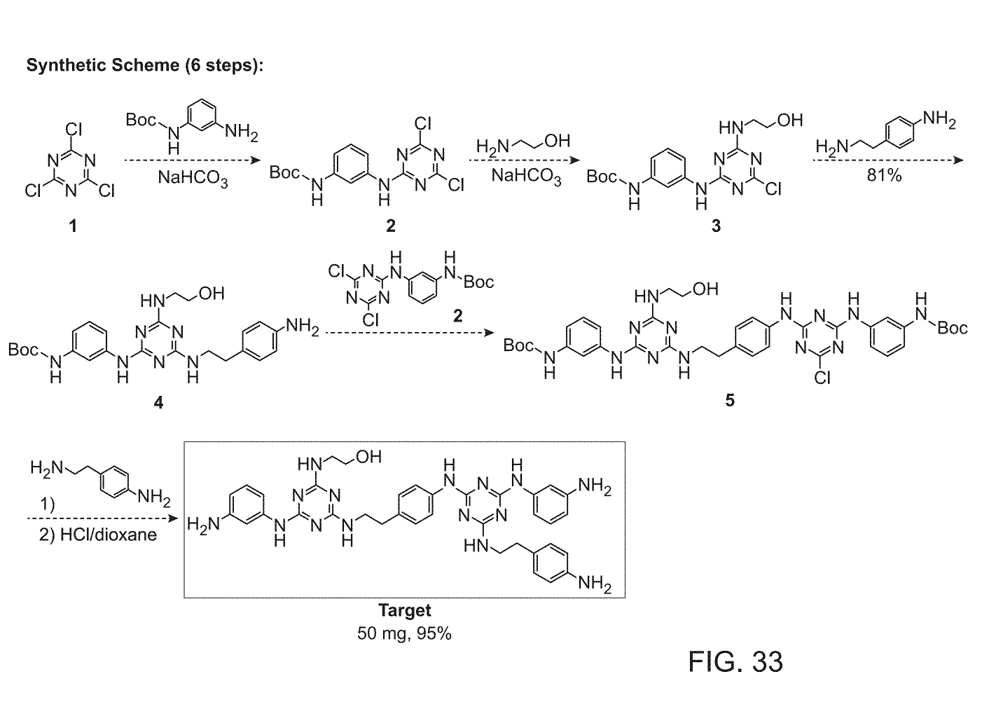

The minimized Z-domain is linked using a flexible peptide linker (GGGGS)_{N} (SEQ ID NO: 17) to an anchor composed of a poly-arginine peptide that binds hyaluronic acid (FIG. 32).

### Example 6

High affinity binders for antibodies have predominately been used for industrial or manufacturing as tools for antibody bioseparation. However, in an alternative usage, as described herein, high affinity binding polypeptides are delivered to a biologic tissue. Such in vivo use of short peptides can be limited by rapid enzymatic degradation. PROSPER (protease specificity prediction server) predicted human enzymatic cleavage sites of peptides used as an anchor. Depicted is SpGc3 (SEQ ID NO:4); protein A Z-domain (SEQ ID NO:3); Protein A minimized Z domain (SEQ ID NO:1) and cysteine stabilized Z domain (SEQ ID NO:2), each of which provides high affinity binding to a therapeutic antibody Fc region. Each can be independently joined to an anchoring domain, exemplified here as LINK (SEQ ID NO:22); or hyaluronic acid binding peptides (SEQ ID NO:23, 24, 25, 26, 27, respectively). Amino acid substitutions with either natural or non-natural amino acids (e.g. beta amino acids, d-amino acids) can occur at or around these cleavage sites to enhance resistance to proteolysis.

These examples are by no means all inclusive, but are meant to highlight that the present disclosure relates to 1) any substance that binds to monoclonal antibody products with a dissociation constant (K_{D}) less than 10⁻⁶ M (K_{D}<10⁻⁶ M), and 2) is anchored in a biologic tissue.

What is claimed is:

1. An anchor molecule, comprising two functional regions or moieties: (1) a region or moiety that binds a therapeutic antibody or fragment thereof with a dissociation constant (K_{D}) less than 10⁻⁶ M, and (2) a region or moiety that anchors the molecule in a biologic tissue of interest; wherein the anchor molecule when delivered to the biologic tissue of interest in combination with a therapeutic monoclonal antibody acts to retain the therapeutic monoclonal antibody in the tissue, thereby increasing the duration of action of the therapeutic monoclonal antibody.

2. The anchor molecule of claim 1, wherein the antibody binding region binds to an Fc region of a therapeutic antibody.

3. The anchor molecule of claim 2, wherein the Fc region is of human IgG class.

4. The anchor molecule of claim 2, wherein the Fc region is of human IgG1 class.

5. The anchor molecule of claim 2, where in the Fc region is recombinant.

6. The anchor molecule of claim 1, where in the antibody binding region or moiety binds to a FAB region of a therapeutic antibody.

7. The anchor molecule of any of claims 1-6, wherein the dissociation constant (K_{D}) is less than 10⁻⁷M.

8. The anchor molecule of any claims 1-6, wherein the dissociation constant (K_{D}) is less than 10⁻⁹ M.

9. The anchor molecule of any claims 1-6, wherein the dissociation constant (K_{D}) is less than 10⁻⁹ M.

10. The anchor molecule of claim 1, wherein the region or moiety that binds a therapeutic antibody is Protein A or a derivative thereof.

11. The anchor molecule of claim 1, wherein the region or moiety that binds a therapeutic antibody is the Z-domain of Protein A or a derivative thereof (SEQ ID NO: 3).

12. The anchor molecule of claim 1, wherein the region or moiety that binds a therapeutic antibody is a minimized form of the Z-domain of Protein A or a derivative thereof (SEQ ID NO: 1).

13. The anchor molecule of claim 1, wherein the region or moiety that binds a therapeutic antibody is a derivative of Protein A that has been stabilized with cysteine residues to prevent degradation and/or enhance binding affinity (SEQ ID NO: 2).

14. The anchor molecule of claim 1, wherein the region or moiety that binds a therapeutic antibody comprises an amino acid sequence based on the binding domain of Protein A (SEQ ID NO: 1, 2, 3, 6, 7, 8, 9, 10, 11, 12, 13, 14).

15. The anchor molecule of claim 1, wherein the region or moiety that binds a therapeutic antibody comprises a small molecule.

16. The anchor molecule of claim 15, wherein the small molecule is based on the binding domain of Protein A or a derivative thereof.

17. The anchor molecule of claim 16, wherein the region or moiety that binds a therapeutic antibody comprises a triazine based small molecule.

18. The anchor molecule of claim 17, wherein the region or moiety that binds a therapeutic antibody comprises a triazine based compound from FIG. 11, 12, 13, 14, 15.

19. The anchor molecule of claim 15, wherein the region or moiety that binds a therapeutic antibody is 1,3-bis({4-[(3-aminophenyl)amino]-6-[(2-hydroxyethyl)amino]-1,3,5-triazin-2-yl}amino)propan-2-ol (compound 1)

20. The anchor molecule of claim 15, wherein the region or moiety that binds a therapeutic antibody is 2-({4-[(3-aminophenyl)amino]-6-({2-[4-({4-[(3-aminophenyl)amino]-6-{[2-(4-aminophenyl)ethyl]amino}-1,3,5-triazin-2-yl}amino)phenyl]ethyl}amino)-1,3,5-triazin-2-yl}amino)ethan-1-ol (compound 2).

21. The anchor molecule of claim 1, wherein the region or moiety that binds a therapeutic antibody is based on Protein G or a derivative thereof.

22. The anchor molecule of claim 21, wherein the derivative of Protein G is based on the C3 domain of Protein G or a derivative thereof (SEQ ID NO: 4)

23. The anchor molecule of claim 1, wherein the region or moiety that binds a therapeutic antibody is based on a non-immunoglobulin scaffold

24. The anchor molecule of claim 23, wherein the non-immunoglobulin scaffold is based on the Sac7d protein or a derivative thereof (SEQ ID NO: 5)

25. The molecule of claim 23, wherein affinity maturation or structure based design is used to enhance binding affinity between the non-immunoglobulin scaffold and the therapeutic antibody.

26. The anchor molecule of any of claims 1-25, wherein the region or moiety that anchors the molecule in a biologic tissue of interest is covalently joined to the region or moiety that binds a therapeutic antibody.

27. The anchor molecule of claim 26, wherein the region or moiety that anchors the molecule in a biologic tissue does so by binding to the biologic tissue of interest

28. The anchor molecule of claim 26, wherein the binding to the biologic tissue of interest is non-covalent.

29. The anchor molecule of claim 28, wherein the binding to the biologic tissue is mediated through a peptide.

30. The anchor molecule of claim 29, wherein the peptide binds to hyaluronic acid.

31. The anchor molecule of claim 30, wherein the peptide is a LINK domain.

32. The anchor molecule of claim 31, wherein the LINK domain comprises the amino acid sequence set forth in SEQ ID NO:22.

33. The anchor molecule of claim 30, wherein the peptide is a Hyaluronic Acid Binding Protein (HABP).

34. The anchor molecule of claim 33, wherein the HABP comprises an amino acid sequence set forth in SEQ ID NO: 23, 24, 25, 26, or 27.

35. The anchor molecule of claim 30, wherein the peptide is a poly-arginine sequence.

36. The anchor molecule of claim 30, wherein the peptide is a poly-lysine sequence.

37. The anchor molecule of claim 29, wherein the peptide binds to type II collagen

38. The anchor molecule of claim 29, wherein the peptide binds type IV collagen.

39. The anchor molecule of claim 30, wherein the peptide binds the alpha-3 subunit of type IV collagen.

40. The anchor molecule of claim 29, wherein the peptide is a non-immunogloblin scaffold.

41. The anchor molecule of claim 40, wherein the non-immunoglobulin has undergone affinity maturation to specifically bind to the biologic tissue of interest.

42. The anchor molecule of claim 28, wherein the region or moiety that anchors the molecule in a biologic tissue of interest comprises one or more guanidinium groups.

43. The anchor molecule of claim 25, wherein the region or moiety that anchors the molecule in a biologic tissue is a polymer.

44. The anchor molecule of claim 41, wherein the polymer is biodegradable.

45. The anchor molecule of claim 44, wherein the biodegradable polymer is hyaluronic acid

46. The anchor molecule of claim 44, wherein the biodegradable polymer is chitosan

47. The anchor molecule of claim 44, wherein the biodegradable polymer is poly (lactic-co-glycolic acid (PLGA)

48. The anchor molecule of claim 41, wherein the polymer is non-biodegradable.

49. The anchor molecule of claim 48, wherein the polymer is poly(methyl methacrylate) (PMMA)

50. The anchor molecule of claim 26, wherein the binding to the tissue of interest is covalent.

51. The anchor molecule of claim 50, wherein the binding to the tissue of interest is mediated through N-Hydroxysuccinimide (NHS).

52. A method for treating a tissue in individual with a therapeutic antibody, the method comprising:
delivering to the tissue an effective dose of an anchor molecule according to any of claims 1-51, in combination with an effective dose of the therapeutic antibody; wherein the duration of action of the therapeutic antibody is increased relative to the duration of action in the absence of the anchor molecule.

53. The method of claim 51, wherein the therapeutic antibody comprises a monoclonal antibody product

54. The method of claim 51, wherein the therapeutic antibody contains a human IgG Fc region.

55. The method of claim 51, wherein the therapeutic antibody contains a human IgG1 Fc region.

56. The method of any claims 51-55, wherein the biologic tissue is the vitreous cavity of the eye.

57. The method of any claims 51-55, wherein the anchor molecule is delivered either before, during, or after delivery of the therapeutic antibody.

58. The method of claim 56, wherein the anchor molecule is delivered through an intraocular injection.


SEQUENCE LISTING (27 sequences)

SEQ ID NO: 1 - protein, 33 aa, Artificial sequence
  synthetic polypeptide
FNMQQQRRFYEALHDPNLNEEQRNAKIKSIRDD

SEQ ID NO: 2 - protein, 34 aa, Artificial sequence
  synthetic polypeptide
FNMQCQRRFYEALHDPNLNEEQRNAKIKSIRDDC

SEQ ID NO: 3 - protein, 58 aa, Artificial sequence [sequence omitted]

SEQ ID NO: 4 - protein, 56 aa, Artificial sequence
  synthetic polypeptide
TTYKLVINGKTLKGETTTKAVDAETAEKAFKQYANDNGVDGVWTYDDATKTFTVTE

SEQ ID NO: 5 - protein, 66 aa, Artificial sequence
  synthetic polypeptide
  misc_feature (7)..(9): Xaa can be any naturally occurring amino acid
  misc_feature (44)..(44): Xaa can be any naturally occurring amino acid
  misc_feature (46)..(46): Xaa can be any naturally occurring amino acid
SVKVKFXXXGEEKEVDTSKIRDVCRQGKNVKFLYNDNGKYGAGXVXEKDAPKELLDMLARAEREKK

SEQ ID NO: 6 - protein, 12 aa, Artificial sequence
  synthetic polypeptide
EPIHRSTLTALL

SEQ ID NO: 7 - protein, 8 aa, Artificial sequence
  synthetic polypeptide
FYWHCLDE

SEQ ID NO: 8 - protein, 6 aa, Artificial sequence
  synthetic polypeptide
HWRGWV

SEQ ID NO: 9 - protein, 8 aa, Artificial sequence
  synthetic polypeptide
  misc_feature (1)..(8): Xaa can be any naturally occurring amino acid
XXXXXXXX

SEQ ID NO: 10 - protein, 12 aa, Artificial sequence
  synthetic polypeptide
CFFHHCFFHHKG

SEQ ID NO: 11 - protein, 16 aa, Artificial sequence
  synthetic polypeptide
RTYRTYRTYRTYKKKG

SEQ ID NO: 12 - protein, 16 aa, Artificial sequence
  synthetic polypeptide
  MISC_FEATURE (1)..(12): The amino acids from positions 1 to 12 are non-natural d-amino acids
RTYRTYRTYRTYKKKG

SEQ ID NO: 13 - protein, 56 aa, Artificial sequence
  synthetic polypeptide
GFRKYLHFRRHLLGFRKYLHFRRHLLGFRKYLHFRRHLLGFRKYLHFRRHLLKKKG

SEQ ID NO: 14 - protein, 68 aa, Artificial sequence
  synthetic polypeptide
VRLGWLLAPADLDARVRLGWLLAPADLDARVRLGWLLAPADLDARVRLGWLLAPADLDARKRGKRGKG

SEQ ID NO: 15 - protein, 5 aa, Artificial sequence
  synthetic polypeptide
GGGGS

SEQ ID NO: 16 - protein, 18 aa, Artificial sequence
  synthetic polypeptide
KESGSVSSEQLAQFRSLD

SEQ ID NO: 17 - protein, 14 aa, Artificial sequence
  synthetic polypeptide
EGKSSGSGSESKST

SEQ ID NO: 18 - protein, 12 aa, Artificial sequence
  synthetic polypeptide
GSAGSAAGSGEF

SEQ ID NO: 19 - protein, 5 aa, Artificial sequence
  synthetic polypeptide
EAAAK

SEQ ID NO: 20 - protein, 7 aa, Artificial sequence
  synthetic polypeptide
  MISC_FEATURE (2)..(6): The amino acid sequence from positions 2 to 6 may be repeated N times
AEAAAKA

SEQ ID NO: 21 - protein, 2 aa, Artificial sequence
  synthetic polypeptide
  MISC_FEATURE (1)..(2): The amino acid sequence from positions 1 to 2 may be repeated N times
  MISC_FEATURE (1)..(1): Xaa may be any amino acid
XP

SEQ ID NO: 22 - protein, 97 aa, Artificial sequence
  synthetic polypeptide
GVYHREARSGKYKLTYAEAKAVCEFEGGHLATYKQLEAARKIGFHVCAAGWMAKGRVGYPIVKPGNCGFGKTGIIDYGIRLNRSERWDAYCYNPHAK

SEQ ID NO: 23 - protein, 11 aa, Artificial sequence
  synthetic polypeptide
RYPISRPRKRC

SEQ ID NO: 24 - protein, 11 aa, Artificial sequence
  synthetic polypeptide
RPSRPRIRYKC

SEQ ID NO: 25 - protein, 27 aa, Artificial sequence
  synthetic polypeptide
LKQKIKHVVKLKVVVKLRSQLVKRKQN

SEQ ID NO: 26 - protein, 15 aa, Artificial sequence
  synthetic polypeptide
STMMSRSHKTRSHHV

SEQ ID NO: 27 - protein, 16 aa, Artificial sequence
  synthetic polypeptide
GAHWQFNALTVRGGGS